(19) United States
(12) Patent Application Publication
(10) Pub. No.: US 2026/0240879 A1
(43) Pub. Date: Aug. 20, 2026

(54) TOPICAL PALA THERAPY FOR CANCER

(71) Applicant: THE CLEVELAND CLINIC FOUNDATION, Cleveland, OH (US)

(72) Inventors: Christine MCDONALD, Cleveland, OH (US); George STARK, Cleveland, OH (US)

(21) Appl. No.: 19/469,498

(22) PCT Filed: Mar. 27, 2024
(86) PCT No.: PCT/US2024/021625

§ 371 Date: Sep. 26, 2025

Related U.S. Application Data
(60) Provisional application No. 63/492,381, filed on Mar. 27, 2023.

(51) Int. Cl. A61K 31/662; A61K 9/00; A61K 9/06; A61K 31/4745; A61K 31/513; A61K 31/5377; A61K 45/06; A61K 47/10; A61K 47/14

(52) U.S. Cl. CPC A61K 31/662; A61K 9/0014; A61K 9/06; A61K 31/4745; A61K 31/513; A61K 31/5377; A61K 45/06; A61K 47/10; A61K 47/14

(57) ABSTRACT

Provided herein are compositions, systems, kits, and methods for applying a composition topically to a bodily area of a subject that has precancerous and/or cancerous cells (e.g., skin cancer cells) and/or is adjacent to underlying precancerous and/or cancerous cells, where the composition comprises N-phosphonacetyl-L-aspartate (PALA) (aka sparfosic acid) and water, and optionally further comprises at least one non-ionic surfactant.

The present application claims priority to U.S. Provisional application Ser. No. 63/492,381, filed Mar. 27, 2023, which is herein incorporated by reference in its entirety.

This invention was made with government support under W81XWH-16-1-0439 awarded by the Department of Defense. The government has certain rights in the invention.

### FIELD OF THE INVENTION

Provided herein are compositions, systems, kits, and methods for applying a composition topically to a bodily area of a subject that has precancerous and/or cancerous cells (e.g., skin cancer cells) and/or is adjacent to underlying precancerous and/or cancerous cells, where the composition comprises N-phosphonacetyl-L-aspartate (PALA) (aka sparfosic acid) and water, and optionally further comprises at least one non-ionic surfactant.

### BACKGROUND

A staggering 1 in 5 Americans will develop skin cancer in their lifetimes¹. Most skin cancers are caused by sun exposure, leading to ultraviolet light (UV)-induced damage². Skin cancers are categorized as either melanoma or non-melanoma (NMSCs)³, which are the most common type of cancers overall and arise in the basal, squamous, and Merkel cells of the skin³. Basal cell carcinoma is the most common, with an estimated 5.4 million diagnoses annually in the United States⁴, followed by squamous cell carcinoma (SCC), the second most common and the most metastatic³. SCC kills more than twice as many individuals as melanoma, at an estimated rate of 15,000 people per year⁵. The majority of SCCs arise from actinic keratoses (AKs), pre-cancerous lesions that affect over 58 million Americans, making AKs one of the most common skin conditions treated by dermatologists⁶. The direct treatment cost of NMSCs in the United States is estimated to be $4.8 billion per year, not including additional indirect costs in lost wages^{7,8}.

Currently, there are a number of FDA-approved therapies for NMSC, each with its own advantages and limitations. The gold standard treatment is surgical removal (e.g. Mohs surgery). For precancers (AKs), the gold standard is cryotherapy. These treatments can be more than 90% effective for individual lesions. However, a major issue not addressed by the localized destructive methods (surgery or cryosurgery) is the fact that most AK lesions arise within a large area of skin that has been damaged by chronic sun exposure and is therefore full of pre-neoplastic cells, a phenomenon called “field cancerization.”

To address the high statistical risk of cancer arising within these regions, several non-invasive topical treatments are currently approved, including fluorouracil (5-FU; an antimetabolite), imiquimod (IMQ; an immunomodulator), and tirbanibulin (a microtubule inhibitor). All three topical treatments are FDA-approved for AK and have reported response rates of 40-80% for AKs, with 5-FU being most effective⁹. 5-FU and IMQ are also FDA-approved to treat superficial BCC, and are often used off-label for superficial SCC treatment. Although each of these topical agents can treat multiple, diffuse lesions (thereby addressing the “field cancerization” problem), and often result in an overall higher rate of long-term clearance than cryotherapy, each is also associated with an intense local skin reaction characterized by pain, burning, skin erosions and, in some instances, hyper- or hypo-pigmentation of the skin. Therefore, there is still a need for improved topical NMSC therapies that are highly effective yet associated with fewer side effects than the currently available options.

### SUMMARY OF THE INVENTION

Provided herein are compositions, systems, kits, and methods for applying a composition topically to a bodily area of a subject that has precancerous and/or cancerous cells (e.g., skin cancer cells) and/or is adjacent to underlying precancerous and/or cancerous cells, where the composition comprises N-phosphonacetyl-L-aspartate (PALA) (aka sparfosic acid) and water, and optionally further comprises at least one non-ionic surfactant. In certain embodiments, the PALA is present in the composition at a concentration of about 0.1% to about 5.0% or 1.0 to 8.0%.

In some embodiments, provided herein are methods of treating precancerous and/or cancerous cells of a subject comprising: applying a composition topically to a bodily area of a subject that has precancerous cells and/or cancerous cells and/or is adjacent to underlying precancerous and/or cancerous cells, or providing the composition to the subject such that the subject is able to apply the composition to the bodily area, wherein the composition comprises N-phosphonacetyl-L-aspartate (PALA) and water, and optionally further comprises at least one non-ionic surfactant.

In certain embodiments, the area of the subject comprises skin (e.g., on face, arm, leg, torso, etc.). In other embodiments, the cancerous cells comprise melanoma and non-melanoma skin cancer cells. In further embodiments, the non-melanoma skin cancer cells are basal cell carcinoma cells or a squamous cell carcinoma cells. In particular embodiments, the precancerous cells comprise actinic keratosis cells (e.g., as part of an actinic keratosis lesion). In particular embodiments, the subject is a female, wherein the area of the subject comprises a surface of the subject's vagina, and wherein the precancerous and/or cancerous cells comprise vaginal precancerous and/or cancer cells. In other embodiments, the area of the subject comprises a surface of the subject's oral cavity (e.g., gums), and wherein the precancerous and/or cancerous cells comprise oral cavity precancerous or cancerous cells. In additional embodiments, the subject is a female, wherein the area of the subject comprises the subject's cervix, and wherein the precancerous and/or cancerous cells comprise cervical precancerous and/or cancerous cells.

In some embodiments, the applying the composition, or providing the composition, is repeated daily for at least 7 days (e.g., 7 . . . 10 . . . 20 . . . or 30 days), for at least 14 days, for at least 21 days, or for at least 7 weeks (e.g., 7, 8, 9, 10 . . . 20 . . . 30 . . . or 40 weeks). In particular embodiments, the applying and providing are repeated at least twice or three times per day for at least 7 or at least 14 days. In certain embodiments, the methods further comprise administering an immune checkpoint inhibitor (e.g., targeting CTLA4, PD-1, or PD-L1; such as pembrolizumab, ipilimumab, nivolumab, or atezolizumab) to the subject. In some embodiments, the subject is a human, or dog, cat, cow, pig, or horse. In particular embodiments, topically administering the composition to an area of a subject with precancerous and/or cancerous cells, at or near the area, reduces or eliminates at least some, or all, of the cancerous or precancerous cells. In particular embodiments, the composition is present in a skin patch or a dispensing device. In certain embodiments, the non-ionic surfactant is selected from the group consisting of: polyglycerol alkyl ethers, glucosyl dialkyl ethers, crownethers, ester-linked surfactants, polyoxyethylene alkyl ethers, Brij, Spans (sorbitan esters) and Tweens (Polysorbates). In particular embodiments, the non-ionic surfactant is selected from the group consisting of: Capryol PGMC, Polysorbate 80, Polysorbate 20, Sodium Lauryl Sulfate, Poloxamer 188, Polyoxyl 40 hydrogenated castor oil, Mannide Monooleate, and Lauroyl polyoxyl-32 glycerides.

In some embodiments, provided herein are systems and kits comprising: a) a composition comprising N-phosphonacetyl-L-aspartate (PALA) and water, and optionally at least one non-ionic surfactant; and b) at least one of the following: i) a dispensing container configured to dispense the composition topically to a bodily area of a subject, and/or ii) a topical patch configured to deliver the composition topically to the bodily area of the subject, and/or iii) an immune checkpoint inhibitor (e.g., targeting CTLA4, PD-1, or PD-L1; such as pembrolizumab, ipilimumab, nivolumab, or atezolizumab).

In certain embodiments, the composition is present in the dispensing container or the topical patch. In other embodiments, the dispensing container is present, and wherein the dispensing container is a touchless dispensing container that allows for dispensing, and rubbing in of, the composition on the bodily area such that other areas of the subject do not come into contact with the composition. In particular embodiments, the other areas of the subject comprises the subject's hands.

In some embodiments, the touchless dispensing container comprises a metered-dose topical applicator. In other embodiments, the touchless dispensing container is selected from the group consisting of: a CLICK Metered Topical Applicator, a TOPI-CLICK applicator, a TAPEMARK unit dose semi-solid drug delivery system, MICROBRISTLE APPLICATOR (MBA™), BACK Easy Lotion Applicator, and LIQUIBAND XL skin closure system. In further embodiments, the dispensing container is present and comprises a tube or sachet.

In some embodiments, provided herein are compositions comprising: a) N-phosphonacetyl-L-aspartate (PALA); b) water; and optionally c) at least one non-ionic surfactant. In certain embodiments, the at least one non-ionic surfactant is present. In certain embodiments, the PALA is present in the composition at a concentration of about 0.1% to about 8.0% (e.g., about 1%, 2%, 3%, 4%, 5%, 6%, 7%, or 8%). In certain embodiments, the composition comprise about 20-50% or 10-75% water.

In some embodiments, the at least one non-ionic surfactant comprises a non-ionic linear copolymer. In further embodiments, the non-ionic linear copolymer comprises poloxamer 188. In other embodiments, the non-ionic linear copolymer is present in the composition at about 50%-90%, about 60%-80%, about 65%-75%, or about 70%. In additional embodiments, the at least one non-ionic surfactant is present in the composition at 10%-30% or about 22%. In additional embodiments, the at least one non-ionic surfactant comprises Caprylocaproyl Polyoxyl-8 glycerides, or wherein the at least one non-ionic surfactant excipient comprises PEG-8 mono- and diesters of caprylic (C8) and capric (C10) acids with a small fraction of mono-, di- and triglycerides, or wherein the at least one non-ionic surfactant comprises LABRASOL ALF.

In additional embodiments, the composition further comprises a hydrogel agent, and the composition is in the form of a hydrogel. In some embodiments, the composition further comprises isopropyl myristate, and optionally wherein the isopropyl myristate is present in the composition at a concentration of about 0.5%-7% or about 1%-5%, or about 3%. In other embodiments, the composition further comprises a solubilizer and/or emulsifier.

In certain embodiments, the composition further comprises macrogolglycerol ricinoleate, and optionally wherein the macrogolglycerol ricinoleate is KOLLIPHOR EL, and optionally wherein the macrogolglycerol ricinoleate is present in the composition at a concentration of about 2%-10% or about 3%-7%, or about 5%. In further embodiments, the composition further comprises at least one non-PALA anti-cancer agent, and optionally wherein the at least one non-PALA anti-cancer agent comprises: tirbanibulin, fluorouracil (5-FU) and/or imiquimod (IMQ). In particular embodiments, the at least one non-ionic surfactant comprises poloxamer 188 and/or Caprylocaproyl Polyoxyl-8 glycerides. In further embodiments, the composition is in the form of a cream, lotion, or gel (and contains the necessary reagents to be in such form).

### DESCRIPTION OF THE FIGURES

FIG. 1: Topical PALA is not toxic up to 5% (w/v) in mice and reduces tumor growth in a dose-dependent manner. A. Percentage weight changes during treatment. B. Levels of serum amyloid A (SAA) at endpoint. C. Colon length at endpoint. D. Levels of fecal lipocalin-2 (LCN2) at endpoint. E. Quantitation of mean tumor numbers per mouse during treatment. F. Change in tumor burden for each mouse during treatment, as a percentage of the original tumor number. Means±SEM, n=4-15 mice/group from 3 independent experiments. Statistical significance determined by mixed effects analysis and post-hoc Bonferroni's multiple comparisons test.

FIG. 2: Daily topical PALA application is better tolerated than NMSC standard-of-care topical treatments. A. Percentage weight changes during treatment. Initiation of supportive care indicated by arrows (T). B. Mean colon lengths on day 7. C. Serum amyloid A levels on day 7. D. Mean spleen weights on day 7. E. Tissue histology of skin treated for 7 days with topical drugs. Means±SEM, n=5-10 mice/group. Statistical significance determined by mixed effects analysis with Bonferroni's multiple comparisons test or one-way ANOVA with Dunnett's multiple comparison test.

FIG. 3: Tumor burdens, tumor areas, and tumor grades are reduced in mice treated with topical PALA. A. Percentage weight changes during treatment. B. Levels of serum amyloid A at endpoint. C. Colon lengths at endpoint. D. Levels of lipocalin-2 (LCN2) at endpoint. E. Mean tumor numbers per mouse during treatment. F. Percentage changes in mean tumor numbers during treatment. G. Quantification of surface tumor areas from photographs during treatment. H. Mean cross-sectional tumor areas, measured from H&E stained tissues. Means±SEM, n=12/group. Significance determined by mixed-effects model (REML) with Bonferroni's multiple comparison test or unpaired t-test.

FIG. 4: Expression of the NOD2 target gene LL-37 is increased by topical PALA treatment. A. Quantitation of the area of DefB14 staining from multiplex immunofluorescent images of skin tumor sections. B. Quantitation of the percentage of LL-37+ cells in tumor regions from multiplex immunofluorescent images of skin tumor sections. C. Representative multiplex immunofluorescent images of skin tumor sections, highlighting differential expression of LL-37 from mice treated with 50% acetone/10% glycerol (vehicle) or 2% (w/v) PALA, stained for LL-37 (green), cytokeratin-14 (CK14, pink), or DNA (blue). Scale bar=2 mm. Higher magnification of boxed area displayed as Inset, with scale bar=1 mm. Means±SEM, n=12-19 tumors/group. Significance determined by unpaired t-test.

FIG. 5: Tumor-infiltrating cell populations are altered by topical PALA treatment, demonstrating immunomodulatory activity. Multiplex immunofluorescent images were quantified for the percentages of tumor cells staining with specific markers. A. Quantitation of cells expressing the neutrophil cell marker myeloperoxidase (MPO; aqua). B. Quantitation of cells expressing the macrophage cell marker F4/80 (pink). C. Quantitation of cells expressing the T cell marker CD3 (red). D. Quantitation of cells expressing the cytotoxic T cell marker CD8 (green). All sections were co-stained with the keratinocyte marker CK14 (grey) and DAPI (blue). E. Representative multiplex immunofluorescent images highlighting differentially present cell types. Means±SEM, n=10-17 tumors/group. Scale bar=2 mm. Significance determined by unpaired t-test.

FIG. 6: Representative photographs of peak skin irritation observed during topical irritation testing. A. Photographs of vehicle, 2% PALA, and 5% IMQ treated mice 3 days post-treatment. B. Photographs of vehicle, 2% PALA, and 5% 5-FU treated mice 7 days post-treatment.

FIG. 7: Topical PALA treatment may induce cell cycle arrest. Representative images of immunostained skin from vehicle and 2% PALA treated mice (left panels). Quantification of the percentage of positive cells within the tumor area is graphed (right panel); mean±SEM, n=12-16 tumors/group. Significance determined by unpaired t-test. Multiplex immunofluorescent images of Ki67 (green) and cytokeratin-14 (CK14; red) stained skin tumor displayed on top row. Immunohistochemistry images show expression of indicated cell cycle markers (brown).

FIG. 8: Topical 2% PALA is non-toxic and shows trends of efficacy in reducing tumor burden in a chemical carcinogen-induced skin cancer model. SKH1-Elite female mice were treated topically with DMBA for 2 weeks and then treated topically with TPA thrice a week for 8 weeks to induce skin cancer. Mice were then randomized into treatment groups and treated topically with vehicle (50% acetone/10% glycerol) or 2% (w/v) PALA for 4 weeks. A. Quantitation of mean change in tumor burden per mouse during the treatment period as a percentage of the tumor number on week 9. B. Weight change during the treatment period presented as percentage of baseline weight. C. Levels of the liver and systemic inflammation marker serum amyloid A (SAA) at endpoint. D. Measurements of colon length at endpoint as a gross measure of intestinal pathology. Means±SEM, n=8-9/group. Significance determined by 2-way ANOVA with Sidak's multiple comparisons test or unpaired two-way t-test. All p values <0.05 are shown on the graphs.

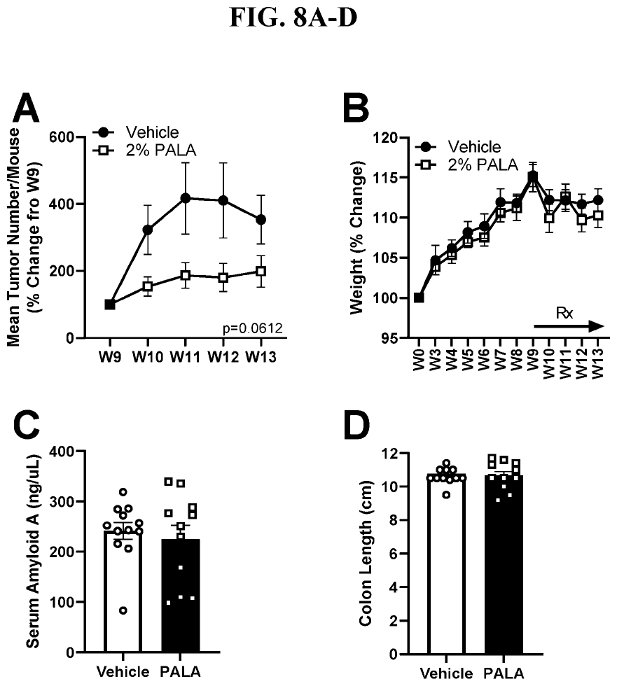

FIG. 9: Topical 2% PALA formulated in Aquaphor is effective in reducing tumor burden in a chemical carcinogen-induced skin cancer model. C57BL/6 female mice were treated topically with DMBA for 2 weeks and then treated topically with TPA thrice a week for 12 weeks to induce skin cancer. Mice were then randomized into treatment groups and treated topically with vehicle (Aquaphor) or 2% (w/v) PALA/Aquaphor for 4 weeks. A. Quantitation of mean change in tumor burden per mouse during the treatment period as a percentage of the tumor number on week 13. B. Quantification of mean change in total tumor area per mouse during the treatment period as a percentage of the week 13 measurements. Means±SEM, n=5-6/group. Significance determined by 2-way ANOVA

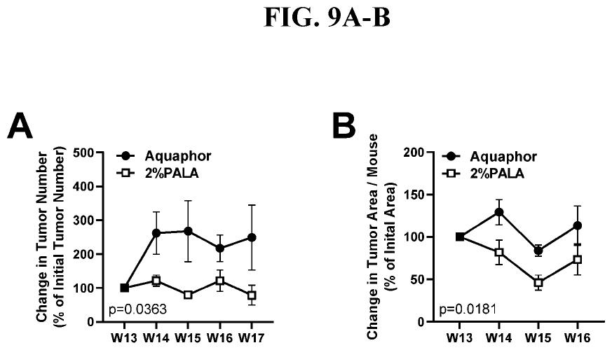

FIG. 10 shows Table 7 from Example 2, which shows the appearance and HPLC assay of different hydrogels.

FIG. 11. Cumulative release profile of different formulations with permeation enhancers (n=1). PALA solution in water is also reported as reference.

FIG. 12 shows Table 10, which shows appearance and assay of formulations from Example 2.

FIG. 13. Cumulative release profile of particular formulations (n=3).

FIG. 14. Skin tissue and plasma levels of PALA over 24 h after a single topical application of formulation AP0536/29/03 4% (w/v) PALA topical to mice. Male CD-1 mice (n=3/time point) had 10 uL of 4% PALA applied to dorsal skin (final dose applied=0.4 mg/kg). Plasma and skin tissue samples were collected at 5 min, 15 min, 30 min, 1 h, 2 h, 4 h, 6 h, and 24 h post-administration and PALA levels determined by LC-MS. Mean±SEM graphed.

FIG. 15. Reformulated topical 2% PALA is non-toxic and effective in reducing tumor burden and total tumor area. SKH1-Elite female mice were UV irradiated thrice a week for 20 weeks to induce skin cancer. Mice were then randomized into treatment groups and treated topically with vehicle (AP0536/29/01), 2% (w/v) PALA (AP0536/29/02), or 4% (w/v) PALA (AP0536/29/03) daily for 6 weeks. A. Weight change during the treatment period presented as percentage of baseline weight. B. Levels of the liver and systemic inflammation marker serum amyloid A (SAA) at endpoint. C. Measurements of colon length at endpoint as a gross measure of intestinal pathology. D. Levels of fecal lipocalin-2 (LCN2) at endpoint as a measure of intestinal inflammation. E. Quantitation of mean tumor burden per mouse during the treatment period. F. Mean change in tumor burden per mouse during the treatment period as a percentage of the original tumor number. G. Quantification of mean tumor surface area per mouse from photographs during the treatment period. Means±SEM, n=8-9/group. Significance determined by mixed-effects model (REML) with Bonferroni's multiple comparisons test or one-way ANOVA with Tukey's multiple comparisons test. All p values <0.05 are shown on the graphs.

FIG. 16. Topical 2% PALA is better tolerated and more effective than two standards of care topical treatments for NMSC. SKH1-Elite female mice were UV irradiated thrice a week for 20 weeks to induce skin cancer. Mice were then randomized into treatment groups (n=5/group) and treated topically with vehicle (AP0536/29/02), 2% (w/w) PALA (AP0536/29/02), 5% (w/w) 5-fluorouracil (5% 5-FU), or 5% (w/w) imiquimod (5% IMQ) daily for 4 weeks. A. PALA treated mice maintain weight is better than 5-FU or IMQ treated mice. Change in weight from the start of treatment graphed (mean±SEM). IMQ and 5-FU mice were provided supportive care (starting at times indicated by †) and 5-FU treated animals were terminated after 2.5 weeks of treatment at the request of veterinary staff. B. Levels of the liver and systemic inflammation marker serum amyloid A (SAA) at endpoint. C. Levels of fecal lipocalin-2 (LCN2) at endpoint as a measure of intestinal inflammation. D. Mice treated with 2% PALA has significantly smaller average tumor size than either vehicle or 5% IMQ treated mice. Quantification of mean tumor surface area per tumor measured from photographs using ImageJ software over the treatment period. E. Growth profile of individual tumors demonstrating a lower percentage of continually growing tumors in 2% PALA treated mice. F. Endpoint tumor grade determined through evaluation of H&E stained tumor cross-sections demonstrating fewer squamous cell carcinoma (SCC) and SCC in situ tumors in 2% PALA treated mice than in either vehicle or 5% IMQ treated mice. G. Tumor levels of interleukin-1β are elevated in both 2% PALA and 5% IMQ treated mice indicating the induction of an anti-tumor immune response. H. Tumor levels of interferon-al are elevated only in 2% PALA treated mice suggesting that the type of immune response elicited is distinct from that stimulated by 5% IMQ.

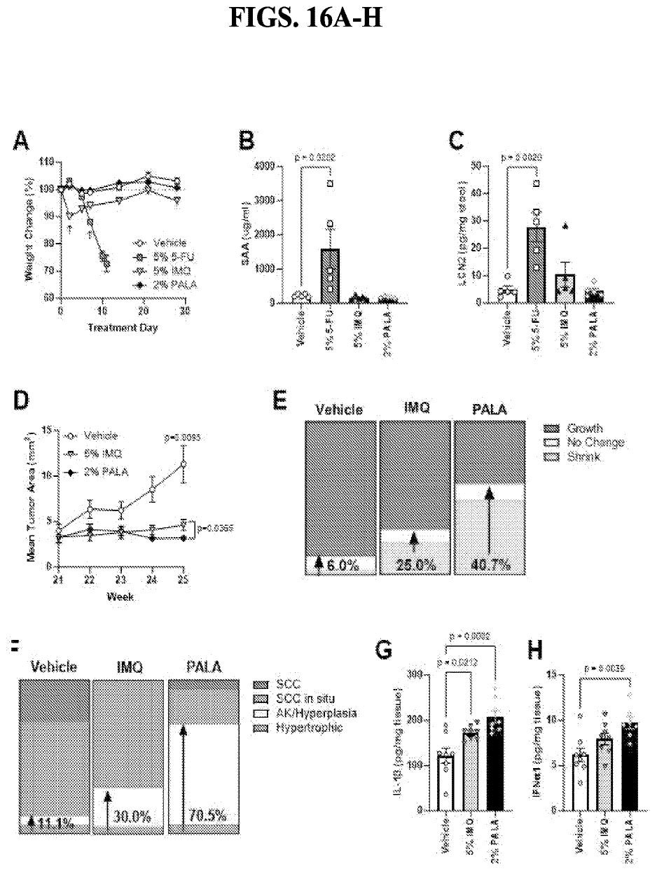

FIG. 17: Topical PALA treatment retards melanoma growth and sensitizes melanoma to anti-checkpoint inhibitor therapy in a B16 melanoma orthotopic xenograft model. C57BU/6 female mice (n=4-5/group) were injected subcutaneously bilaterally with B16 melanoma cells (10⁵ cells/flank; 8-10 injections total). Tumor volume was measured by caliper every 2-3 days by personnel blinded to treatment group. Data from palpable tumors are graphed as mean±SEM. A. Topical treatment with 1% PALA daily for 12 days retards tumor growth. On day 12 post-injection, mice were treated topically with either vehicle (AP0536/29/02) or 1% (w/w) PALA (AP0536/29/02) daily for 12 days. Significant differences were determined by Mixed-effects Model (REML) with Sidak's multiple comparison test. **p<0.01 B. Short term topical treatment of B16 melanoma with 2% PALA reduces expression of the checkpoint inhibitor PD-L1. On day 7 post-injection, mice were treated topically with either vehicle (AP0536/29/02) or 2% (w/w) PALA (AP0536/29/02) daily for 5 days. Tumors were harvested on day 14 post-injection and tumor lysates analyzed by immunoblot for PD-L1 protein levels and quantified by image densitometry using ImageJ relative to tubulin levels (loading control). Mean±SEM graphed. C. Short term topical treatment with 2% PALA sensitizes B16 melanoma to anti-PD-L1 therapy. On day 7 post-injection, mice were treated topically with either vehicle (AP0536/29/02) or 2% (w/w) PALA (AP0536/29/02) daily for 5 days. Mice were also treated with either isotype or anti-PD-L1/atezolizumab antibodies (100 μg/mouse) every 48 hours intraperitoneally from day 7 to day 19. Significant differences were determined by 2-way ANOVA with Dunnett's multiple comparison test. ***p<0.001

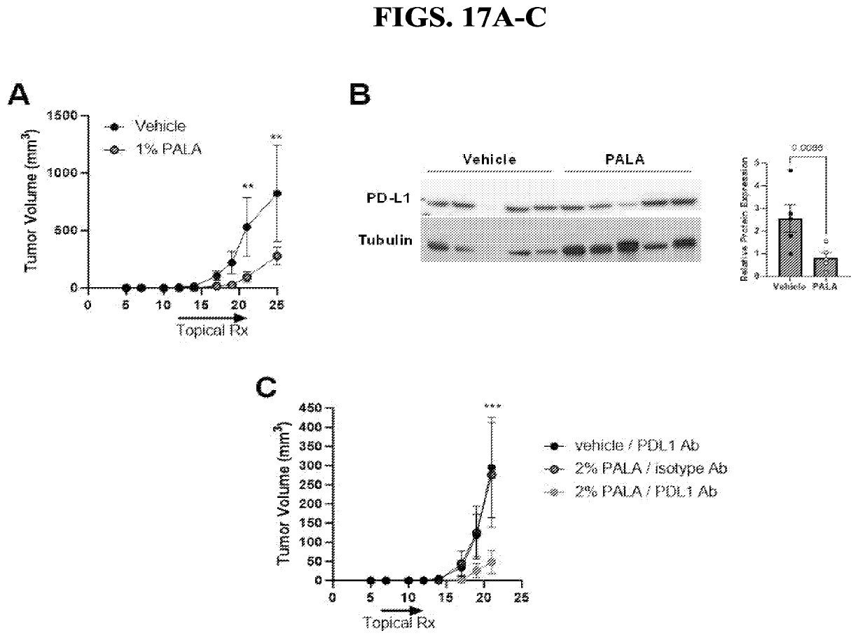

FIG. 18: Tumor growth of oral squamous cell carcinoma orthotopic xenografts is retarded by topical PALA treatment. C57Bl/6 mice (males and females; n=6/group) were injected with MOC-1 squamous cell carcinoma cells in the tongue. Once palpable tumors formed, mouse tongues were treated topically 5×/week with vehicle (AP0536/29/02) or 2% (w/w) PALA (AP0536/29/02). Tumor volume was measured by caliper every 2-3 days. Data are graphed as mean±SEM. Significant differences were determined by Mixed-effects Model (REML) with Sidak's multiple comparison test. *p<0.05, ****p<0.0001.

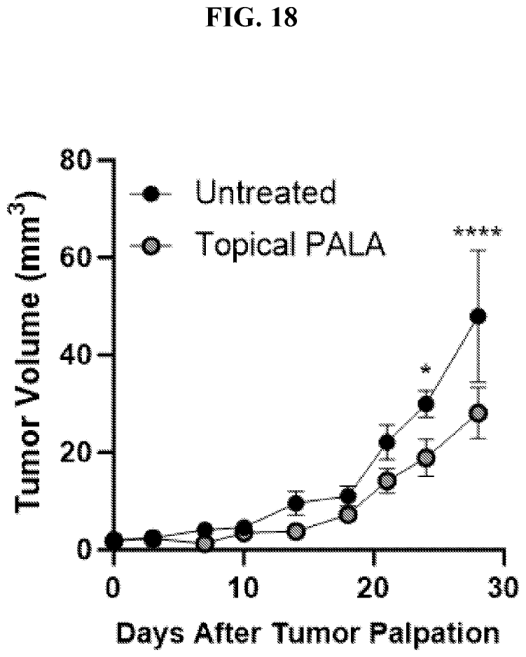

FIG. 19: PALA has a U-shaped dose-response curve and stimulates biological effects independent of enzyme inhibition. A. Bactericidal activity of normal human dermal fibroblasts stimulated by PALA treatment. Cells stimulated for 16 h with the indicated concentration of PALA. Conditioned supernatants added to log phase cultures of methicillin-resistant Staphylococcus aureus (MRSA) for 2 h and viable bacterial determined by selective plating. Mean±SD graphed. B. PALA-stimulated production of antimicrobial peptides in normal human dermal fibroblasts. Cells were stimulated as in A. and supernatants assayed by ELISA for human β-defensin 2 (HBD2) and human β-defensin 3 (HBD3). Mean±SD graphed. C. Enzyme activity of aspartate transcarbamylase, the enzymatic target of PALA, is only reduced ~25% at doses that induce maximal immune stimulation in normal human dermal fibroblasts. Cell lysates from the cells shown in A. were assayed for their ability to catalyze the formation of carbamyl aspartate (CA) in vitro. Percent change in CA levels graphed; mean±SD. D. & E. Similar U-shaped dose-response curves observed in response to topical PALA treatment of skin cancer in the UVB-induced NMSC mouse model. D. Average number of tumors per mouse at the end of the treatment period (mean±SEM graphed). D. Mice treated for 10 weeks with vehicle (Aquaphor), 1%, 2%, or 5% PALA in Aquaphor. E. Mice treated for 4 weeks with vehicle (AP0536/29/02), 2% (w/w) PALA (AP0536/29/02), or 4% (w/w) PALA (AP0536/29/02).

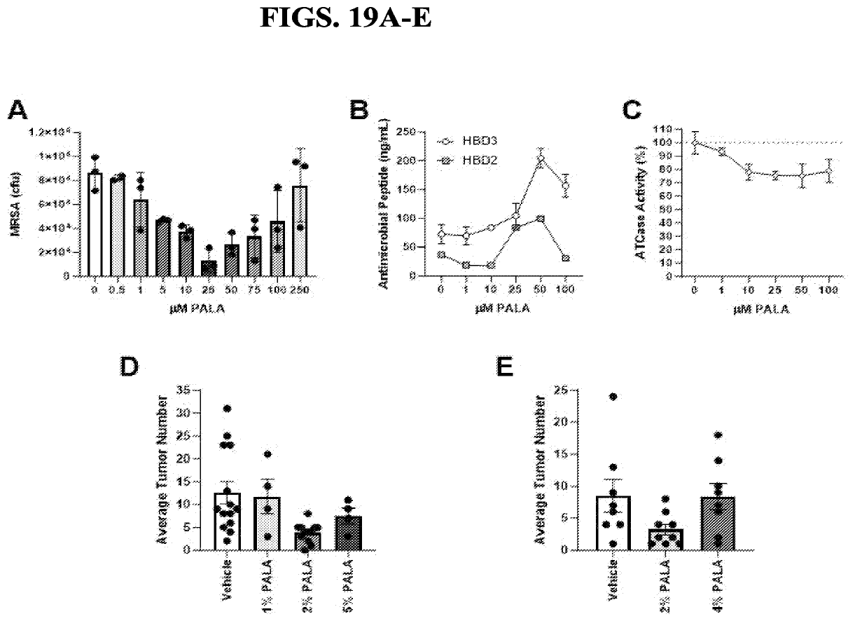

### DETAILED DESCRIPTION

Provided herein are compositions, systems, kits, and methods for applying a composition topically to a bodily area of a subject that has precancerous and/or cancerous cells (e.g., skin cancer cells) and/or is adjacent to underlying precancerous and/or cancerous cells (e.g., in the form of lesions or tumors), where the composition comprises N-phosphonacetyl-L-aspartate (PALA) (aka sparfosic acid) and water, and optionally further comprises at least one non-ionic surfactant. In certain embodiments, the PALA is present in the composition at a concentration of about 0.1% to about 5.0%, or about 1% to about 8% (e.g., about 2%, 4%, 6%, or 8%).

Phosphonacetyl-L-aspartate (PALA) is a potent and highly specific inhibitor of the multi-enzyme protein carbamyl phosphate synthetase II/aspartate transcarbamylase/dihydroorotase (CAD), which catalyzes the first three steps of de novo pyrimidine nucleotide biosynthesis. PALA was designed as a transition-state analog inhibitor of aspartate transcarbamylase, and has nanomolar potency¹⁰. It is readily taken up by cells in culture and kills them efficiently by starving them for pyrimidine nucleotides, as aspartate transcarbamylase is required for this de novo biosynthetic pathway. PALA is quite specific, since the salvage pyrimidine precursor uridine completely prevents its toxicity. Systemic administration of PALA showed great anti-neoplastic efficacy as a single agent in murine B16 melanoma and Lewis lung carcinoma tumor models, but clinical trials of PALA as a systemically administered single agent in humans for colon cancer, breast cancer, malignant melanoma, or advanced soft-tissue sarcoma were disappointing due to limited efficacy¹¹, perhaps, at least in part, because of reversal of pyrimidine synthesis inhibition by dietary uridine.

In certain embodiments, the compositions herein (comprising the PALA) are formulated in a format selected from the group consisting of a cream, a lotion, a spray, an ointment, a gel (e.g., hydrogel), a paste, and a foundation. In other embodiments, such compositions are present in a patch. In particular embodiments, the compositions may include one or more additives selected from the group consisting of: a perfume, colorant, thickening agent, vegetable oil, emulsifier, solvent, pH adjusting agent, antiseptic agent, preservative, vitamin, sun-block, surfactants and combinations thereof. Various physical sunscreen agents such as titanium dioxide, silicone-treated titanium dioxide, zinc oxide, ferrous oxide, ferric chloride, talc, chromium oxide, or cobalt oxides may be included. Alternatively or in addition, a chemical sunscreen agent such as para-amino benzoic acid, esters of para-amino benzoic acid, salicylates, cinnamates, benzophenones, dihydroxyacetone, parsol 1789, or melanin may be included.

Compositions for such topical administration to a subject can be formulated in pharmaceutical compositions. Such pharmaceutical composition, besides containing PALA and water, may contain additional agents. It is not intended that the present invention be limited by the particular nature of the pharmaceutical preparation. For example, such compositions can be provided together with physiologically tolerable liquid, gel or solid carriers, diluents, adjuvants and excipients. These therapeutic preparations can be applied topically to mammals for veterinary use, such as with domestic animals, and clinical use in humans.

### EXAMPLES

### Example 1

### Topical N-Phosphonacetyl-L-Aspartate is a Dual Action Candidate for Treating Non-Melanoma Skin Cancer (NMSC)

This Example describes investigation of topical application of PALA as a NMSC therapy, by combining the chemotherapeutic and immune modulatory features of 5-flurouracil and imiquimod. Daily topical application of PALA to mouse skin was well tolerated and resulted in less irritation, fewer histopathological changes, and less inflammation than caused by either 5-flurouracil or imiquimod. In an ultraviolet light-induced NMSC mouse model, topical PALA treatment substantially reduced the numbers, areas, and grades of tumors, compared to vehicle controls. This anti-neoplastic activity was associated with increased expression of the antimicrobial peptide LL-37 and increased recruitment of CD8⁺ T cells and F4/80+ macrophages to the tumors, demonstrating both immunomodulatory and anti-proliferative effects.

### Materials and Methods

### Reagents

PALA (NSC-224131) was obtained from the National Cancer Institute (NCI)/Division of Cancer Treatment and Diagnosis (DCTD)/Developmental Therapeutics Program (DTP) Open Chemical Repository. PALA was dissolved at concentrations of 1%, 2% or 5% (w/v) in 50% acetone/10% glycerol (Fisher Scientific, Hampton, New Hampshire). Aquaphor Healing Ointment was purchased from Beiersdorf USA (Wilton, Connecticut). Imiquimod Cream (Aldara, 5%; NDC #45802-368-62) was purchased from Perrigo Company (Allegan, Michigan). Fluorouracil Cream USP (Efudex, 5%, NDC #51672-4118-6) was purchased from Taro Pharmaceutical Industries Ltd (Hawthorne, New York).

### UVB-Induced NMSC Mouse Model

Animal procedures were approved by the Institutional Animal Care and Use Committee at Cleveland Clinic (IACUC protocol #2018-2096) and performed in accordance with relevant institutional and national guidelines for the care and use of laboratory animals. Anesthetized female SKH1-Elite mice (Crl:SKH1-Hr^{hr}, strain code 477, Charles River Laboratories) were exposed to UVB thrice a week for 20 weeks as previously described¹². The head and tail regions were masked with felt during UVB exposure to limit tumor induction to only dorsal skin. A custom UV lamp unit equipped with ten 7.2W G8T5 bulbs (305 nm peak wavelength) with an average irradiance between 0.312-0.356 mW/cm² (USHIO America, Inc.) was used for UVB exposure. The UVB irradiance was measured prior to each exposure session using a PMA2100 radiometer equipped with UVA and UVB detectors (Solar Light) and the time required to achieve the desired dose calculated. Dose of UVB was gradually increased (10% per week) over the first 10 weeks starting at 80 mJ/cm² dose to a final dose of 175 mJ/cm² (time range of 8 min 11 s-9 min 20 s, depending on UVB irradiance measurements).

After 20 weeks of UVB exposure, mice were divided based on initial number of tumors into treatment groups to balance the initial tumor burden. Mice were treated daily with 200 μL of drug, topically applied to dorsal skin for 10 weeks. Mice were co-housed in small groups (n=5/cage) and oral exposure of the drug was minimized by the use of a rapidly absorbed vehicle (50% acetone/10% glycerol). At weekly intervals, the mice were weighed and their body condition score was assessed. Tumor burdens were determined weekly from in-person counts and digital photographs quantitated by using ImageJ software¹³. Individual tumors were numbered and tracked during image analysis; if tumor boundaries between individual tumors became confluent (e.g. 2 tumors merge), it was still counted as 2 tumors and the area averaged.

At harvest, colon lengths were measured and blood collected in K2-EDTA microtainers (Becton Dickinson) then processed into plasma for serum amyloid A (SAA) measurement by ELISA. All skin lesions were counted at harvest and tumors large enough to be bisected were collected. Tumor tissues were bisected and half was fixed in Histochoice, and embedded in paraffin blocks, and the other half was frozen for ELISA analysis. Tissue sections were stained with hematoxylin/eosin for histopathologic assessment or underwent multiplex immunofluorescent imaging. Skin collected from the mice with UV-induced tumors was carefully selected to bisect the largest tumors for staining. In cases where the mice had several tumors, not every tumor or lesion could be excised. Therefore, we prioritized larger, more pronounced tumors in all the mice and examined as many tumors as possible. The tissues examined by the resident dermatologist were not representative of the whole sample and some smaller precancerous lesions were excluded; however, this was a consistent practice among all treatment groups.

### Skin Irritation Testing

The topical formulations of 2% PALA/50% acetone/10% glycerol, 5% imiquimod (IMQ), 5% 5-FU (5-FU), vehicle (50% acetone/10% glycerol) control, and Aquaphor were applied (~0.05 g/mouse) daily to the dorsal skin of SKH1-Elite female mice (8 weeks of age; n=5/group) for 7 days. The Aquaphor and vehicle treated groups were combined during final analyses into a single group, as there were no differences detected between these groups. Mice were monitored daily for weight change, skin appearance, and signs of distress. In response to significant weight loss in the IMQ and 5-FU treatment groups, these mice were supplemented with Diet Gel 76A (ClearH2O) starting on day 3 (IMQ) or day 5 (5-FU). At harvest, spleen weights and colon lengths were measured. Blood was collected in K2-EDTA microtainers (Becton Dickinson) and processed into plasma for SAA measurement by ELISA. Skin tissue was harvested and either fixed in Histochoice and embedded in paraffin blocks, or frozen for ELISA analysis. Tissue sections were stained with hematoxylin/eosin for histopathologic assessment.

### Enzyme-Linked Immunoassays

Serum amyloid A levels were measured in plasma samples using the Mouse SAA ELISA kit (E-90SAA, Immunology Consultants Laboratory, Inc.) according to manufacturer's instructions. Lipocalin-2 (LCN2) was quantified from pre-weighed stool samples homogenized in 0.5 mL PBS using the Mouse Lipocalin-2/NGAL DuoSet ELISA (DY1857) and DuoSet Ancillary Reagent Kit 2 (DY008, R&D Systems). Skin protein lysates were made by homogenizing frozen tissue with a pestle and scissors in RIPA buffer (50 mM Tris, pH8, 150 mM NaCl, 0.5% sodium deoxycholate, 1% NP-40, 0.1% sodium dodecyl sulfate, lx Pierce protease inhibitor cocktail (A32965, ThermoFisher)). Cytokines and chemokines were quantified from 25 μg of lysate using a custom 10-plex U-PLEX panel (K15069L-2; Meso Scale Discovery) that included: GM-CSF, KC/CXCL1, IFNα, IFNβ, IFNγ, IL-10, IL-6, IL-12p70, IL-17A, IL-17C, IL-17F, IP-10/CXCL10, MCP-1/CCL2, RANTES/CCL5, and TNFα. The MESO SECTOR S 600 plate reader (ICOAA-0; Meso Scale Discovery) was used to collect the U-Plex data and data was analyzed using MSD Discovery Workbench 4.0 software.

### Multiplex Immunofluorescent Imaging

Immunohistochemistry staining was performed using the Discovery ULTRA automated stainer from Roche Diagnostics (Indianapolis, IN). In brief, antigen retrieval was performed using Proteinase K (IHC Select, 21627; Millipore) and/or a Tris/Borate/EDTA buffer (Discovery CC1, 06414575001; Roche), pH 8.0 to 8.5. Antigens were denatured using a citrate buffer (Discovery CC2, 05424542001; Roche). Time, temperatures, and dilutions are listed in Tables 13-15.

TABLE 13
Antimicrobial Peptide Multiplex Staining Panel Conditions

| | | | Primary | | Opal |
|---|---|---|---|---|---|
| | | Antigen | Incu- | Primary | Fluoro- |
| Antibody | Clone | Retrieval | bation | Dilution | phore |
| Defensin | rabbit | CC1 | 60 min | 1:200 | 570 |
| Beta 14 | polyclonal | 16 min | RT | | 1:1000 |
| LS Bio | aa23-67 | 95° C. | | | FP148801 |
| LS-C318990 | epitope | | | | |
| LL-37 | rabbit | CC1 | 36 min | 1:800 | 620 |
| LS Bio | polyclonal | 16 min | RT | | 1:1000 |
| LS-C190698 | aa140-173 | 95° C. | | | FP1495001 |
| | epitope | | | | |
| Cytokeratin | rabbit | CC1 | 36 min | 1:2000 | 690 |
| 14 | monoclonal | 16 min | RT | | 1:1500 |
| Abcam | EPR17350 | 95° C. | | | FP1497001 |
| ab181595 | | | | | |

TABLE 14
Immune Cell Multiplex Staining Panel Conditions

| | | Antigen | Primary | Primary | Opal |
|---|---|---|---|---|---|
| Antibody | Clone | Retrieval | Incubation | Dilution | Fluorophore |
| F4/80 | rat | Proteinase K | 36 min | 1:100 | 570 |
| Abcam | monoclonal | 8 min | 37° C. | | 1:1000 |
| ab6640 | CI:A3-1 | RT | | | FP148801 |
| Myeloperoxidase | rabbit | Proteinase K | 36 min | 1:200 | 480 |
| Abcam | polyclonal | 8 min RT & | 37° C. | | 1:800 |
| ab9535 | | CC1 | | | FP1500001 |
| | | 24 min 95° C. | | | |
| CD3 | rabbit | Proteinase K | 16 min | predilute | 620 |
| Roche | monoclonal | 8 min RT & | 37° C. | | 1:1000 |
| 05278422001 | 2GV6 | CC1 | | | FP1495001 |
| | | 24 min 95° C. | | | |
| CD8 | rabbit | Proteinase K | 20 min | predilute | 520 |
| Roche | monoclonal | 8 min RT & | 37° C. | | 1:1000 |
| 05937248001 | SP57 | CC1 | | | FP1487001 |
| | | 24 min 95° C. | | | |
| Cytokeratin 14 | rabbit | CC1 | 36 min | 1:2000 | 690 |
| Abcam | monoclonal | 16 min | RT | | 1:1500 |
| ab181595 | EPR17350 | 95° C. | | | FP1497001 |

TABLE 15
Cell Proliferation Multiplex Staining Panel Conditions

| | | Antigen | Primary | Primary | Opal |
|---|---|---|---|---|---|
| Antibody | Clone | Retrieval | Incubation | Dilution | Fluorophore |
| Ki67 | rabbit | CC1 | 60 min | 1:1500 | 570 |
| Novus Biologicals | monoclonal | 56 min | RT | | 1:1000 |
| NB110-89717 | | 95° C. | | | FP488001 |
| Cytokeratin 14 | rabbit | CC1 | 36 min | 1:2000 | 690 |
| Abcam | monoclonal | 56 min | RT | | 1:1500 |
| ab181595 | EPR17350 | 95° C. | | | FP1497001 |

The antibodies were visualized using the OmniMap anti-Rabbit HRP (05269679001; Roche), and UltraMap anti-Rat HRP (05891884001; Roche) in conjunction with the Akoya Biosciences (Marlborough, MA) Opal Fluorophores, listed with their respective antibodies (Tables 1-3). The slides were counterstained with Spectral DAPI (FP1490; Akoya Biosciences).

For cell secreted molecules (e.g. DefB14), the areas of positive staining within tumor tissue were measured using QPath software. First, the total tissue area was assessed (both tumor and non-tumor) using a “tissue detection” command. Next, a thresholder command was used to determine the amount of specific fluorophore detected within the region. A percentage of area covered by the targeted fluorophore was calculated for each sample.

Within the same annotation of tissue regions, the proportion and densities of LL37⁺ cells, CD3⁺ T cells, CD8⁺ T cells, F4/80⁺ macrophages, and MPO⁺ cells were assessed using a positive cell detection method on the QuPath software. This allows for a thresholder function to be used to determine cellular radius and fluorophore detection threshold. DAPI or an auto-fluorescent detection channel were used to determine total number of cells in the tumor region. A percentage of positive cells for each targeted fluorophore was calculated for each detection channel.

### Immunohistochemical Staining

Immunohistochemical staining was performed using the Discovery ULTRA automated stainer from Roche Diagnostics by the Cleveland Clinic Imaging Core. In brief, antigen retrieval was performed using a Tris/borate/EDTA buffer (Discovery CC1, #06414575001, Roche), pH 8.0 to 8.5. Time, temperatures, and dilutions are found in table 16.

TABLE 16
Cell Cycle Marker Staining Conditions

| | | Antigen | Primary | Primary |
|---|---|---|---|---|
| Antibody | Clone | Retrieval | Incubation | Dilution |
| Cyclin A2 | rabbit | CC1 | 60 min | 1:500 |
| Abcam | monoclonal | 32 min | RT | |
| ab181591 | EPR17351 | 95° C. | | |
| Cyclin B1 | rabbit | CC1 | 60 min | 1:500 |
| Abcam | monoclonal | 32 min | RT | |
| ab181593 | EPR17060 | 95° C. | | |
| Cyclin D1 | rabbit | CC1 | 60 min | 1:100 |
| Abcam | monoclonal | 32 min | RT | |
| ab16663 | SP4 | 95° C. | | |
| p-Histone H3 | rabbit | CC1 | 60 min | 1:100 |
| Cell Signaling | polyclonal | 16 min | RT | |
| Technologies | | 95° C. | | |
| 9701L | | | | |

The antibodies were visualized using the OmniMap anti-rabbit HRP (#05269679001, Roche) in conjunction with the ChromoMap DAB detection kit (#05266645001, Roche). Lastly, the slides were counterstained with hematoxylin and bluing.

### Statistical Analyses

Statistical analyses were performed on GraphPad Prism software (Version 9.1). One-way or two-way Analysis of Variance (ANOVA) with post-hoc Bonferroni or Tukey's multiple comparison test was used to determine significance of datasets with multiple variables. Chi-squared test was applied for categorical variable comparisons. Details of tests used for specific experiments have been included in the figure legends.

### Results

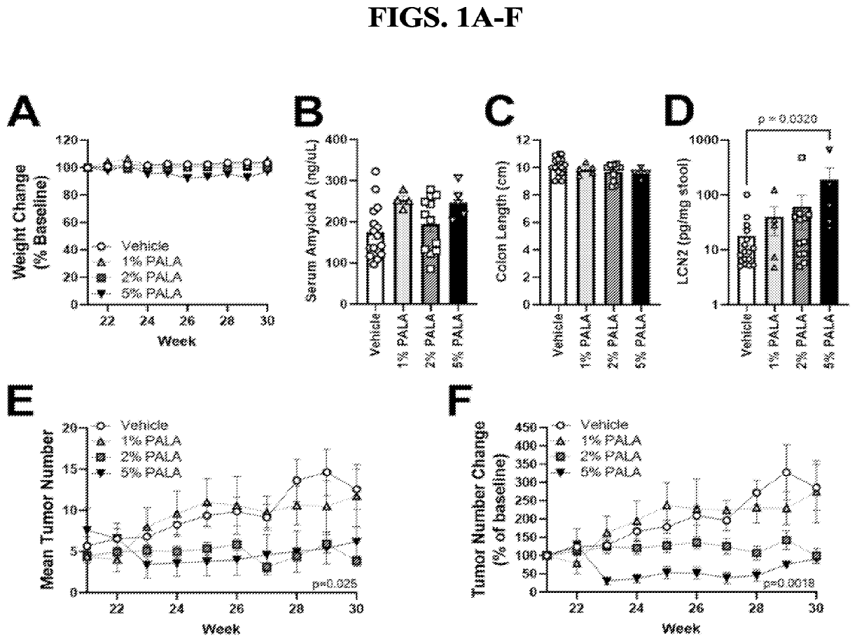

Topical PALA is not toxic up to 5% (w/v) and reduces tumor growth in a dose-dependent manner. To determine an optimal dose of topical PALA for treatment of NMSC, SKH1-Elite mice were UV-irradiated thrice a week for 20 weeks to induce SCCs and then treated daily for 10 weeks with 0, 1, 2, or 5% (w/v) PALA in 50% acetone/10% glycerol. Topical PALA treatment was well tolerated over 10 weeks, with no apparent skin irritation or weight loss (FIG. 1A). Previous toxicology studies identified the intestine, liver, and central nervous system as the first targets of systemic PALA toxicity¹¹; therefore, circulating levels of serum amyloid A (SAA) were measured from plasma collected at endpoint as a marker of liver inflammation and systemic toxicity (FIG. 1B). No significant differences were found between treatment groups; however, there were trends of higher SAA levels in the 1% and 5% PALA-treated mice (p=0.067 and p=0.066, respectively). Colons were examined for gross pathology and colon lengths were measured at harvest as a quantitative measure of intestinal pathology. None of the mice had any apparent gross colon pathology or significant differences in colon lengths between treatment groups (FIG. 1C). Levels of fecal lipocalin-2 (LCN2) were also quantified as a marker of intestinal inflammation. Only mice treated with 5% PALA had significantly higher LCN2 levels as compared to vehicle treated mice (186.8±121.1 vs. 18.1±6.0, p=0.032; FIG. 1D). Importantly, mice treated with 2% or 5% PALA had substantially fewer total tumors (3.83±0.6 and 6.20±1.8, respectively at endpoint) over the 10-week treatment period than those treated with either vehicle or 1% PALA (12.5±2.4 and 11.7±3.8, respectively at endpoint, p=0.029; FIG. 1D). This was especially apparent when the average change from initial tumor burden was calculated; the vehicle- and 1% PALA-treated groups showed steady increases in average tumor burden (286.4%±62.6 and 275.0%±84.7 of original size at endpoint, respectively) that was not observed in the 5% PALA group (91.6%±13.4 of original size at endpoint) as early as 3 weeks of treatment and by 8 weeks of treatment for the 2% PALA group (99.2%±20.5 of original size at endpoint, p=0.0018; FIG. 1E). These results indicate that topical PALA is well tolerated up to 5% (w/v) over 10 weeks of daily treatment and demonstrate anti-tumor activity at concentrations of 2% and higher. The dose of 2% PALA was selected for further analysis, based on a significant anti-neoplastic effect (mean tumor burden change of 286.4%±62.6 in vehicle vs. 99.2%±20.5 of original size for 2% PALA at endpoint, p=0.0018) and the absence of any elevation of the inflammatory markers SAA and LCN2 at this dose.

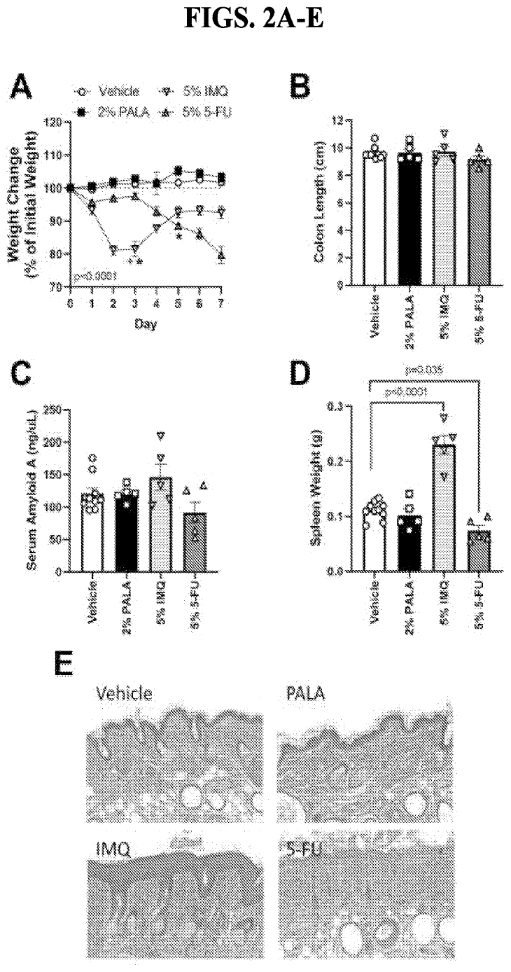

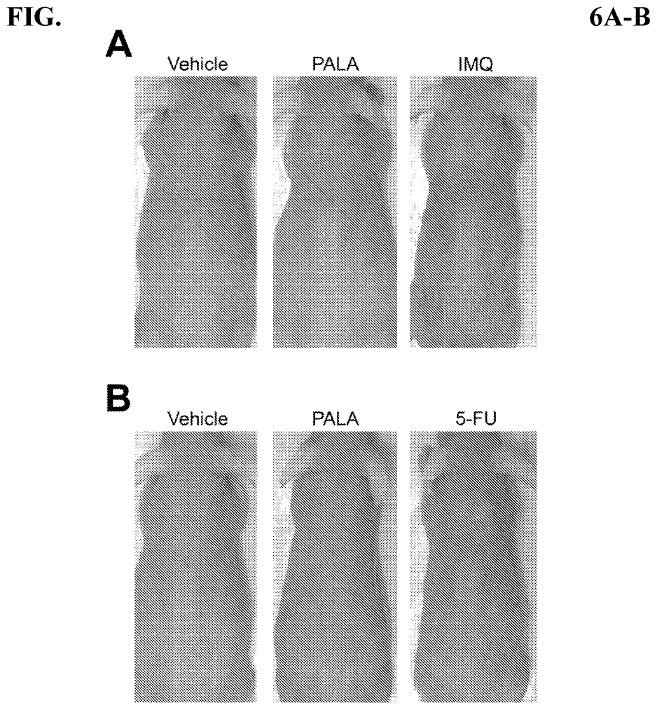

Daily application of topical PALA is better tolerated than NMSC standard-of-care topical treatments. The tolerability of daily treatment with the current standard of care topical ointments, 5% IMQ and 5% 5-FU were compared to 2% PALA on naïve SKH1-Elite mice, as well as their vehicle controls, over 7 days. Skin appearance, weight change, and body condition score were assessed daily. In contrast to vehicle and 2% PALA treated mice, significant weight loss was observed in mice treated topically with either IMQ or 5-FU, requiring interventional care and resulting in one death (FIG. 2A, arrows). Skin irritation was apparent on IMQ treated mice as early as day 2 and day 5 in the 5-FU group; no skin irritation was seen in either vehicle or 2% PALA treated mice (FIG. 6).

Upon harvest on day 7, gross pathology of the spleen and intestine were assessed, SAA levels measured, as well as skin histopathology and expression of inflammatory mediators in treated skin evaluated. No differences in gross intestinal pathology or SAA levels were observed among groups (FIGS. 2B & 2C). Similar to previous reports of topical IMQ treatment of mice¹⁴, mice treated with IMQ had splenomegaly (FIG. 2D), overt thickening of the epidermis (acanthosis; FIG. 2E), and upregulation of IL-17 family cytokines in the treated skin tissue (Table I).

TABLE I
Differential cytokine/chemokine profile of 2%
PALA vs. standard of care topical treatments.

| Cytokine/ | Vehicle | 2% PALA | 5% IMQ | 5% 5-FU |
|---|---|---|---|---|
| Chemokine | Mean (SEM) | Mean (SEM) | Mean (SEM) | Mean (SEM) |
| IL-6 | 25.4 (3.2) | 38.8 (8.1) | 61.3 (10.0) | 268.8 (104.8) |
| | | | | p = 0.0131 |
| IL-17A | 0.6 (0.1) | 0.5 (0.09) | 1.6 (0.1) | 1.2 (0.4) |
| | | | p = 0.0177 | |
| IL-17C | 0.5 (0.03) | 0.6 (0.1) | 1.0 (0.04) | 1.4 (0.1) |
| | | | p = 0.0122 | p < 0.0001 |
| KC | 26.3 (6.4) | 31.1 (2.8) | 28.6 (5.9) | 114.5 (16.4) |
| | | | | p = <0.0001 |
| RANTES | 43.7 (3.2) | 46.4 (9.6) | 141.0 (36.1) | 17.7 (5.0) |
| | | | p = 0.0061 | |
| GM-CSF | 0.8 (0.1) | 1.0 (0.1) | 2.5 (0.7) | 2.6 (0.2) |
| | | | p = 0.0265 | p = 0.0188 |

Significance determined by one-way ANOVA with Dunnett's multiple comparison test, n = 5/group. The following cytokines and chemokines were profiled but not differentially expressed: TNFα, IFNα, IFNβ, IL-1β, IL-12p70, IL-17F, MCP-1, and IP-10.

In addition, cutaneous levels of RANTES and GM-CSF were elevated in IMQ treated animals (Table I). 5-FU-treated mice also demonstrated toxicity characterized by significant decreases in spleen weight (FIG. 2D), epithelial erosion (FIG. 2E), and increased expression of IL-6, IL-17C, KC, and GM-CSF in treated skin lysates (Table I). In contrast, PALA-treated mice had no changes in spleen weight, colon length, SAA levels, skin histology, or expression of an inflammatory cytokine/chemokine panel in treated skin as compared to vehicle controls. These findings indicate that daily application of topical PALA to mice is much better tolerated than current standard of care topical treatments for NMSC.

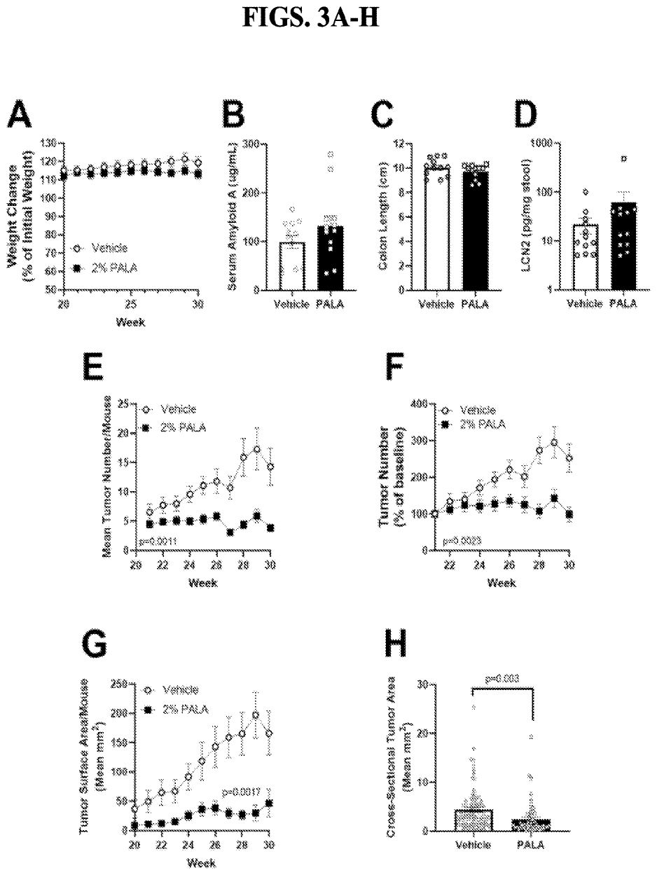

Tumor burden, tumor area, and tumor grade are reduced in mice treated with topical PALA. AK lesions and SCC tumors were induced on SKH1-Elite mice through exposure to UVB over 20 weeks. Tumor bearing mice were treated daily with either 2% PALA or vehicle control daily for 10 weeks and evaluated for toxic side effects of treatment, as well as for treatment-induced changes in tumor number, size, and grade. Daily treatment of mice with topical PALA for 10 weeks was well tolerated as reflected by no significant differences in weight, SAA levels, gross intestinal pathology, or LCN2 levels as compared to vehicle-treated control mice (FIGS. 3A-D). PALA-treated mice had a significantly lower tumor burden than vehicle-treated mice; the average number of tumors on vehicle-treated mice increased over the 10-week treatment period (endpoint value of 252.3%±34.3, p=0.00051), while the average tumor number per mouse in the PALA-treated group remained constant (endpoint value of 99.2%±20.5, p=0.97) (FIGS. 3E & 3F). Tumor sizes were also dramatically larger in the vehicle-treated group, both for mean surface tumor area (endpoint values of 197.1 mm² vs. 30.3 mm²; p<0.0001) and for mean tumor cross-sectional area (endpoint values of 4.4 mm² vs. 2.4 mm²; p=0.003) (FIGS. 3G & 3H). When the growth of individual tumors were analyzed, a greater proportion of PALA treated tumors resolved over the treatment period than vehicle controls (83.3% vs. 26.8%, respectively, p<0.0001; Table II).

TABLE II
Individual tumor growth phenotypes

| | | Vehicle^{a} | PALA^{a} | |
|---|---|---|---|---|
| | | (n = 41) | (n = 48) | p-value^{b} |
| Tumor | Larger | 24 (58.5%) | 6 (12.5%) | <0.0001 |
| Growth | Same | 3 (7.3%) | 0 (0%) | |
| Outcome | Smaller | 3 (7.3%) | 2 (4.1%) | |
| | Resolved | 11 (26.8%) | 40 (83.3%) | |
| Tumor | Continual Growth | 19 (46.3%) | 1 (2.0%) | <0.0001 |
| Growth | Grow then Shrink | 12 (29.2%) | 33 (68.7%) | |
| Phenotype | No Change | 3 (7.3%) | 0 (0%) | |
| | Continual Shrinkage | 7 (17.0%) | 14 (29.1%) | |

^{a}Data presented as N (%)
^{b}Based on Chi-squared test for categorical variables comparisons

Additionally, the growth phenotype was distinct between the treatments, with most of the PALA treated tumors shrinking after an initial period of growth or continually reducing in size (97.9%) vs. the continual growth observed in the vehicle treated animals (46.3%; p<0.0001; Table II). These data support the use of topical PALA for the treatment of, for example, AK and SCC lesions.

Excised tumors were histologically graded as either benign/hyperplastic lesions, AK/SCC in situ, well differentiated SCC, or poorly differentiated SCC (Table III).

TABLE III
Tumor differences by treatment and tumor grade

| Characteristics^{a} | Tumor Grade | Vehicle | PALA | p-value^{b} |
|---|---|---|---|---|
| Total Tumor Number | Poorly Differentiated SCC | 25 | 6 | 0.0002 |
| | Well Differentiated SCC | 37 | 12 | |
| | AK/SCC in situ | 28 | 23 | |
| | Benign/ Hyperplastic | 0 | 8 | |
| Tumors / Mouse | Poorly Differentiated SCC | 2.0 (1.6) | 0.5 (0.9) | 0.0002 |
| | Well Differentiated SCC | 3.0 (1.8) | 1.0 (1.1) | |
| | AK/SCC in situ | 2.3 (2.1) | 1.9 (1.2) | |
| | Benign/ Hyperplastic | 0 (0) | 0.5 (0.6) | |
| Tumor Prevalence | Poorly Differentiated SCC | 28% | 13% | <0.0001 |
| | Well Differentiated SCC | 41% | 25% | |
| | AK/SCC in situ | 31% | 49% | |
| | Benign/ Hyperplastic | 0% | 13% | |
| Tumor Area | Poorly Differentiated SCC | 59.5 (77.3) | 62.6 (70.0) | 0.88 |
| | Well Differentiated SCC | 10.7 (18.0) | 7.5 (4.5) | |
| | AK/SCC in situ | 3.4 (6.6) | 2.3 (2.0) | |
| | Benign/ Hyperplastic | 0 (0) | 1.4 (0.3) | |
| Tumor Cross | Poorly Differentiated SCC | 9.2 (5.8) | 9.8 (6.4) | 0.62 |
| Sectional Area | Well Differentiated SCC | 3.7 (2.0) | 3.1 (2.2) | |
| (mm²) | AK/SCC in situ | 1.5 (0.7) | 1.1 (1.0) | |
| | Benign/ Hyperplastic | n/a | 1.7 (2.0) | |

^{a}Data presented as mean (SEM)
^{b}Based on Chi-squared test for categorical variables comparisons or 2-way ANOVA

Importantly, in comparison to vehicle-treated controls, PALA-treated mice had a lower prevalence of well differentiated SCC and poorly differentiated SCC (38% vs. 69%, p<0.0001) and an increased proportion of AK/SCC in situ and benign lesions (62% vs. 31%, p<0.001), indicating that PALA treatment can block tumor progression.

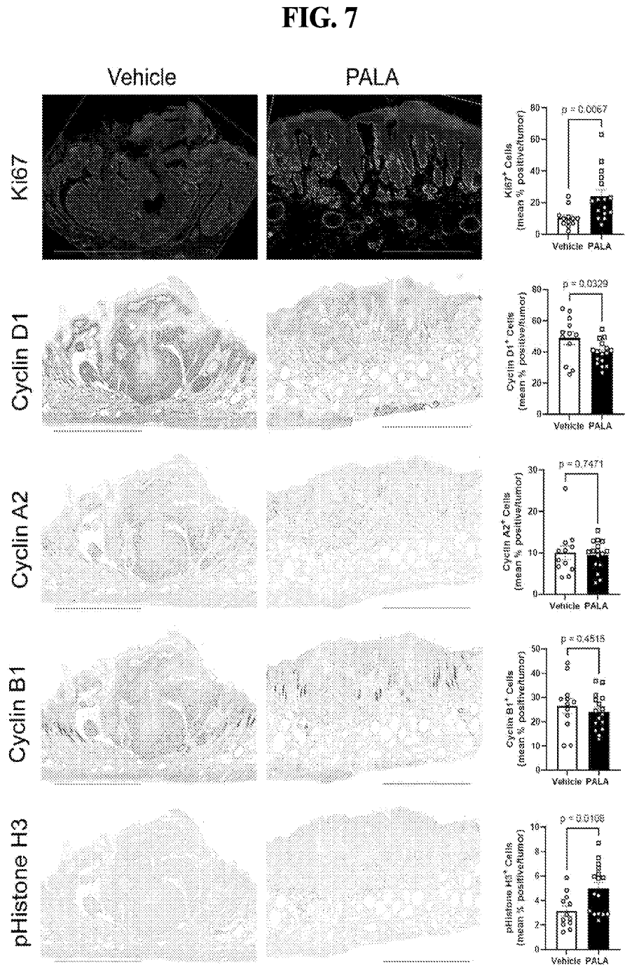

Excised tumors were also stained for the expression of cell cycle markers to determine whether PALA may be inducing cell cycle arrest, similar to 5-FU. No differences were observed in cyclin B1⁺ or cyclin A2⁺ cell populations (FIG. 7). A small elevation in the number of phospho-histone H3⁺ cells was detected in PALA treated samples as compared to vehicle controls (4.9% vs. 3.1%, p=0.0067), but as the total number of positive cells were a very small proportion of the tissue (<5%) and located at the base of tumors, it is unclear whether these cells are an integral component of the tumor and/or exposed to significant amounts of the drug. However, PALA-treated tumors contained a higher average number of Ki67⁺ cells per tumor than vehicle-treated controls (24.6% vs. 10.4%; p=0.005) and a lower percentage of cyclin D1⁺ cells per tumor (39.4% vs. 48.9%, p=0.0329; FIG. 7), suggesting that these tumor cells may be undergoing cell cycle arrest in response to PALA treatment¹⁵.

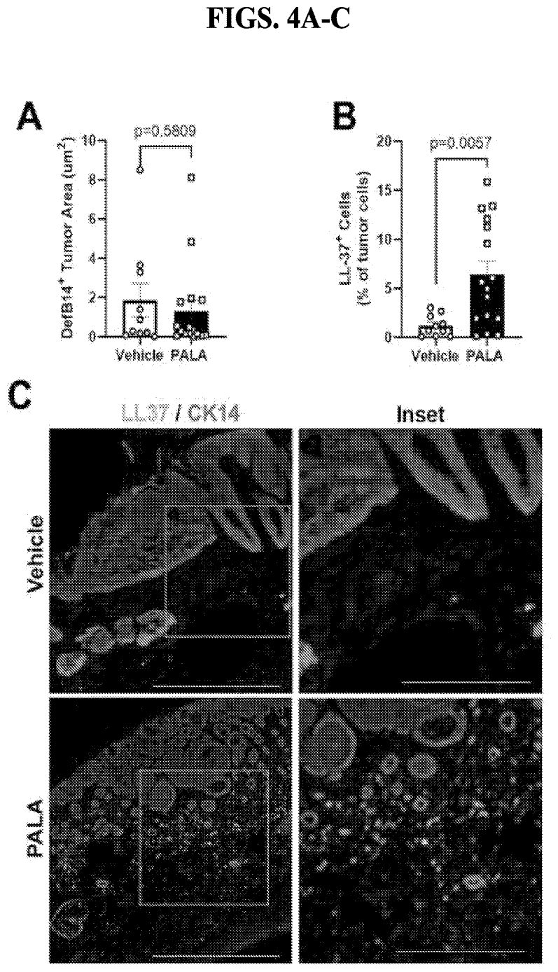

Innate immune activation and enhanced recruitment of cytotoxic T cells are stimulated by topical PALA treatment. Prior work demonstrated that topical PALA treatment of bacterially infected human skin explants stimulated the expression of antimicrobial peptides, such as human β-defensin 2 (HBD2) and cathelicidin (LL-37), in a NOD2-dependent manner¹⁶. Therefore, the levels of the mouse homologues of these AMPs (DefB14 and LL-37) were assessed in the tumor tissues of topically treated mice by multiplex immunofluorescent microscopy. DefB14 staining within tumors was diffuse, indicating AMP secretion from the cells, and the total area of DefB14 staining was not different among treatment groups (FIG. 4A). In contrast, LL-37 staining was discrete and co-localized with nuclear DAPI staining, indicating that the numbers of cells expressing LL-37 should be quantified in the tumor tissues. The results demonstrate that LL-37⁺ cells were significantly increased in the PALA-treated tumor samples (FIG. 4B).

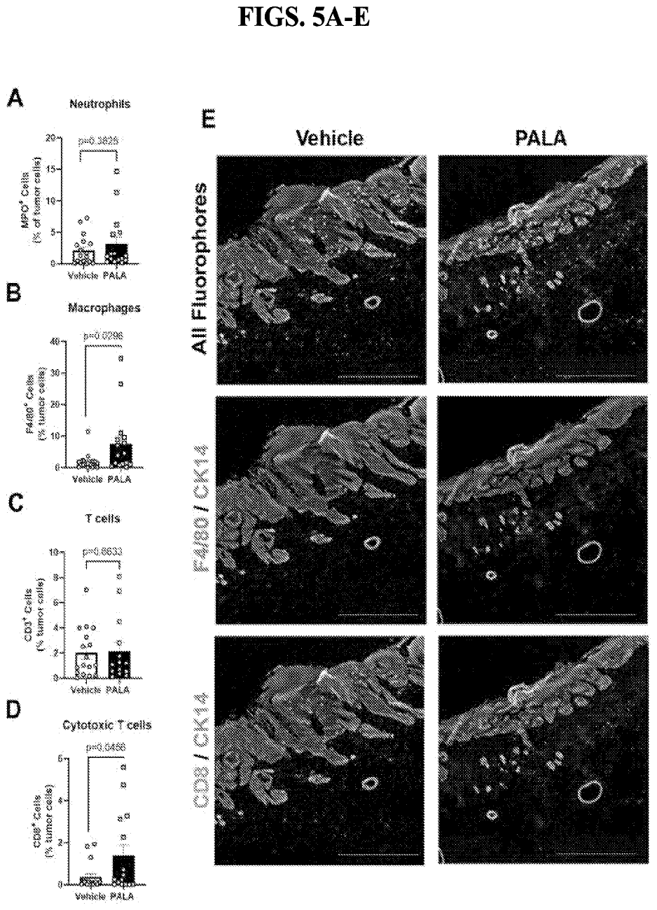

Neutrophils and monocytes/macrophages are major innate immune cell types that express both NOD2 and LL-37^{17,18}. Therefore, tumor sections were analyzed by multiplex immunofluorescent microscopy for markers of neutrophils (myeloperoxidase; MPO) and macrophages (F4/80) to determine whether either of these immune cell populations were recruited to tumor tissues in response to topical PALA treatment. The numbers of MPO⁺ cells were the same between treatment groups, indicating that neutrophils are not differentially recruited to tumor tissue (FIG. 5A). Instead, PALA-treated tumors had increased numbers of F4/80⁺ macrophages relative to vehicle-treated controls (FIGS. 5B & 5E).

LL-37 has a controversial role in carcinogenesis. In some cancers it is upregulated and stimulates the generation of anti-inflammatory tumor-associated macrophages (TAMs) that promote tumorigenesis, while in other cancer types LL-37 promotes the activation and expansion of anti-tumoral CD8⁺ cytotoxic T cells¹⁸. To better understand the role of the LL-37⁺ cells and F4/80⁺ macrophages in PALA-treated tumors, the prevalence of CD8⁺ cytotoxic T cells was assessed by multiplex immunofluorescent microscopy. The overall number of CD3⁺ T cells within tumor tissue was the same between vehicle- and PALA-treated samples (FIG. 5C). However, PALA-treated tumors had elevated numbers of CD8⁺ cytotoxic T cells, compared to vehicle-treated controls (FIGS. 5D & E). Taken together, the results suggest that topical PALA treatment not only inhibits tumor proliferation, but also activates an innate immune response that results in an anti-tumoral cytotoxic T cell response.

This Example demonstrates that topically applied PALA is a new treatment for actinic keratosis and squamous cell carcinoma of the skin. PALA is a simple molecule that can treat cancer through two distinct mechanisms^{16,19-21}. We have successfully shown that daily topical treatment with 2% PALA not only decreases tumor growth but also hinders progression of the tumor to more metastatic cell types. Multiplex immunofluorescent microscopy of the treated tumors showed changes in markers of cell cycle progression and increases in immune regulation corresponding to what is known about the mechanisms of action of this drug¹⁹.

PALA has been extensively tested as a systemic treatment for cancers previously^{22,23}. Although treatments were quite effective in two murine models of colon cancer, the drug could not be used to treat human malignancies due to dose limiting toxicity²³. At low concentrations, we have seen no evidence that the PALA causes any toxicity. We propose that topical treatment with PALA will have advantages over IMQ, 5-FU, and tirbanibulin; current standards of care for NMSC that all cause severe skin irritation and inflammation²⁴. PALA did not induce an inflammatory response or skin irritation when applied to mice topically for up to 10 weeks. PALA is also not chemically reactive and can be stored at ambient temperature, and is therefore a viable option for use in areas with few economic resources²⁰.

While the present disclosure is not limited to any particular mechanism, and an understanding of the mechanism is not necessary to practice the invention, our results with topical PALA suggest that it may induce an immunotherapeutic effect without the need for an additional component for therapeutic action, Other studies investigated the potential of NOD2 activating agents as cancer immunotherapies and found they primarily act as immune adjuvants. Derivatives of the NOD2 bacterial ligand muramyl dipeptide (MDP) conjugated to lipid moieties have demonstrated promising anti-tumorigenic effects in animal models, as well as in human clinical trials²⁵. Direct injection of lipid conjugates of MDP into fibrosarcoma, hepatocellular carcinoma, B16-F10 melanoma, or intravenous administration in a UV-induced skin cancer model or multiple metastatic liver cancer models prevented tumor growth, metastasis, and/or improved survival of the cancer-bearing animals^{25,26}. Although promising results were observed in early phase clinical trials, only two MDP derivatives (Mifamurtide/Mepact® and ImmTher®) have successfully completed phase III trials and gained approval for use in the European Union (Mifamurtide/Mepact®) or were granted FDA approval as an orphan drug (ImmTher®) for non-metastatic osteosarcoma after resection in combination with multidrug chemotherapy²⁷. It is thought that the therapeutic effects are primarily mediated through an adjuvant effect of these MDP/lipid immunotherapies that results in the enhancement of tumoricidal activity of macrophages^{25,28}, a mechanism that is potentially similar to our topical PALA formulation. Recently, a muropeptide produced from Enterococcus faecium was described to augment checkpoint inhibitor cancer therapies in a NOD2-dependent manner²⁹. Similar to our findings with topical PALA, this muropeptide increased anti-tumoral cytotoxic CD8⁺ T cells, as well as impacted the macrophage population.

Both IMQ and PALA are immunomodulators that induce anti-tumoral immune responses. IMQ is a nucleoside in the imidazoquinoline family and is often used to treat NMSCs^{24,30}. It activates anti-tumor immunity through stimulation of TLR7 in macrophages and other immune cells^{24,30}. IMQ is known to upregulate the expression of interferon-α and interleukins 1, 6, and 8, as well as trigger autophagic cell death in macrophages^{24,30,31}. We have shown that PALA treatment does not increase these cytokines, and that macrophages are more abundant in the treated areas, showing that PALA works through an immune mechanism different from that activated by IMQ. While the present invention is not limited to any particular mechanism, and an understanding of the mechanism of the present invention is not necessary to practice the present invention, it is postulated that PALA may be inducing anti-tumoral responses through enhanced cross-presentation of tumoral antigens to result in increased tumor-specific, cytolytic activity of CD8⁺ T cells. This hypothesis is supported by studies describing enhanced NOD2-dependent MHC class I and MHC class II antigen cross-presentation in cells stimulated with MDP³²⁻³⁴, but remains to be formally tested. Immune cell infiltration is seen in most malignancies³⁵. Macrophages are a major component of solid cancers and can promote tumorigenesis by stimulating angiogenesis, immunosuppression, invasion, and metastasis^{36,37}. Tumor-associated macrophages (TAMs) are key regulators of the connection between the immune system and cancer³⁸. TAMs can fuel, rather than limit, tumor progression, and can negatively impact responses to therapy and suppress T cell recruitment³⁸. However, activated macrophages are effective as a cancer immunotherapy, as they can kill cancer cells directly or indirectly through recruitment of other immune cells, such as cytotoxic T lymphocytes³⁹. Within tumors, T cells often become dysfunctional⁴⁰, and activated macrophages may be able to revive their cytolytic activity³⁹. Recent research highlights how the relationship between TAMs and the tumor microenvironment may lead to improved cancer therapies⁴¹⁻⁴² Again, while the present invention is not limited to any particular mechanism, and an understanding of the mechanism is not necessary to practice the invention, our findings suggest that PALA may recruit and locally activate macrophages to enhance a cytotoxic T cell response.

PALA inhibits pyrimidine nucleotide synthesis and inhibits cellular proliferation through depletion of dCTP and dTTP, leaving treated cells unable to complete DNA synthesis²⁰. Although the expression of Ki-67 is often used as a marker of actively cycling cells, it is only absent from quiescent cells in G0 and may be highly expressed in cells arrested in other phases of the cell cycle²⁷. Cyclin D1 expression peaks at the end of G1 and is required to transition cells into S phase. Given the known impact of PALA on cellular pyrimidine pools²⁰, the increased number of Ki-67⁺ cells and decreased percentage of cyclin D1⁺ cells in PALA-treated tumors supports the conclusion that the cells may be undergoing arrest. These findings highlight the dual function of this drug as a cytostatic and immunomodulatory agent.

### Example 2

### PALA Formulations

This Example describes the development of a topical formulation for PALA (CMI-34-1). As a first approach several hydrogels were prepared using natural and synthetic polymers at different concentrations: xanthan gum, methylcellulose, hydroxyethylcellulose, carbopol 971P NF, poloxamer 188 and 407. Poloxamer 188 20% w/w resulted in the only one providing PALA permeability in in vitro diffusion test (Franz cells). As a second approach, a few permeation enhancers (transcutol HP, labrasol ALF and isopropyl myristate) were added to the poloxamer 188 hydrogel with the aim of improving PALA permeability. In vitro diffusion test confirmed that labrasol ALF clearly improved the PALA permeability, therefore, it was included in the formulation. To refine the formulation, isopropyl myristate and kolliphor EL were added in the formulation for their emollient and texture enhancement properties. Additionally, they also have a permeation enhancement role. Even though they did not improve the permeation in vitro, they might play a role in vivo. A formulation including P188 20% w/w (70%), labrasol ALF (21.6%), kolliphor EL (5.4%), and isopropyl myristate (3%) was selected as formulation mouse studies. Stability studies conducted at RT and refrigerated (2-8° C.) conditions for 9 weeks confirmed the stability of the formulation in both conditions.

### Details of HPLC Method

Standard solutions of PALA (CMI-34-1) were prepared in diluent (100% Type I water) at concentration of 1 mg/mL, 20 mg of PALA were accurately weighed in a 20 mL volumetric flask and the volume adjusted with 100% Type I water. The samples were sonicated for 20 minutes until complete dissolution and injected into HPLC for system suitability testing. To assess precision and repeatability, standard A was injected 5 times and standard B was injected twice, the main peak area and % relative standard deviation (% RSD) were calculated. The % RSD of the main peak of the API for standard solutions must be <2.0%. according to internal guidelines. Finally, the standard agreement was calculated as described by the following equation:

$$standard agreement = \frac{{Area}_{A}}{{Area}_{B}} \times \frac{{Conc}_{B}}{{Conc}_{A}}$$

Where Area_{A} and Area_{B} is the area under the peak of standards A and B, respectively and Conc_{A} and Conc_{B} is the concentration of the standards A and B, respectively.

### Preparation of Placebo Hydrogels

Placebo hydrogels were prepared as in Table 4.

TABLE 4
List of placebo hydrogels

| Polymer | Composition | | Preparation procedure |
|---|---|---|---|
| Xanthan gum | 2% | w/v | Xanthan gum was accurately weighed |
| | 1% | w/v | in a glass beaker, and the volume was |
| | | | adjusted with type I water at room |
| | | | temperature. The beaker was placed on |
| | | | a stirrer and left stirring at room |
| | | | temperature until a clear solution was |
| | | | obtained. |
| Methyl- | 2% | w/v | Methylcellulose was accurately |
| cellulose | 1% | w/v | weighed in a glass beaker and finely |
| | | | dispersed with type I water pre-heated |
| | | | at approx. 80° C. A spatula was used to |
| | | | stir the suspension for approx. 30 |
| | | | seconds and the beaker was left in the |
| | | | fridge overnight (for 12 hours). The |
| | | | morning after a clear solution was |
| | | | obtained. |
| Hydroxyethyl- | 2% | w/v | Hydroxyethylcellulose was accurately |
| cellulose | 0.5% | w/v | weighed in a glass beaker, and the |
| | | | volume was adjusted with type I water |
| | | | at room temperature. The beaker was |
| | | | placed on a stirrer and left stirring at |
| | | | room temperature until a clear solution |
| | | | was obtained. |
| Carbopol 971P | 2% | w/w | Carbopol 971P NF was accurately |
| NF | | | weighed in a glass beaker, and the |
| | | | volume was adjusted with type I water |
| | | | at room temperature. The beaker was |
| | | | placed on a stirrer and left stirring at |
| | | | room temperature until a clear solution |
| | | | was obtained. |
| Poloxamer 188 | 20% | w/w | Poloxamer 188 was accurately weighed |
| (P188) | 34% | w/w | in a glass beaker and the volume was |
| | 40% | w/w | adjusted with type I water at room |
| | | | temperature. A spatula was used to stir |
| | | | the suspension for approx. 30 seconds |
| | | | and the beaker was left in the fridge |
| | | | overnight (for 12 hours). The morning |
| | | | after a clear solution was obtained. |
| Poloxamer 407 | 20% | w/w | Poloxamer 407 was accurately weighed |

| | | |
|---|---|---|
| (P407) | | in a glass beaker and the volume was |
| | | adjusted with type I water at room |
| | | temperature. A spatula was used to stir |
| | | the suspension for approx. 30 seconds |
| | | and the beaker was left in the fridge |
| | | overnight (for 12 hours). The morning |
| | | after a clear solution was obtained. |
| Poloxamer | 10% w/w P188 | Poloxamer 188 and 407 were accurately |
| 188/407 | 15% w/w P407 | weighed in a glass beaker and the |
| (P188/407) | 75% w/w water | volume was adjusted with type I water |
| | | at room temperature. A spatula was |
| | | used to stir the suspension for approx. |
| | | 30 seconds and the beaker was left in |
| | | the fridge overnight (for 12 hours). The |
| | | morning after a clear solution was |
| | | obtained. |

### Preparation of Formulations for In Vitro Testing (Franz Cells)

All the formulations were prepared by accurately weighing 50 mg PALA in a 4 mL glass vial. To this 950 mg of vehicle (listed in table 5) was added. The vial was stirred at RT until a clear solution was obtained. Exception for poloxamers P407 20% w/w and P188 at 40% w/w where the PALA solubilisation was performed by stirring the vial in an ice bath (to prevent gelling which occurred at RT).

TABLE 5
List of formulations tested in Franz cells

| | | PALA |
|---|---|---|
| | | concen- |
| | | tration |
| Formulation | | in the |
| code | Vehicle composition | formulations |
| AP0536/4/01 | 100% P188 (20% w/w) aq. Solution | 50 mg/g |
| AP0536/7/04 | 100% P188 (40% w/w) aq. Solution | 50 mg/g |
| AP0536/13/01 | 100% P188 (34% w/w) aq. Solution | 50 mg/g |
| AP0536/4/05 | 100% P407 (20% w/w) aq. Solution | 50 mg/g |
| AP0536/7/03 | 100% P407 + P188 (15% w/w + | 50 mg/g |
| | 10% w/w) aq. Solution | |
| AP0536/4/02 | 100% carbopol 971P NF (2% w/w) aq. | 50 mg/g |
| | Solution | |
| AP0536/4/03 | 100% hydroxyethylcellulose (2% w/v) aq. | 50 mg/g |
| | Solution | |
| AP0536/7/05 | 100% hydroxyethylcellulose (0.5% w/v) | 50 mg/g |
| | aq. Solution | |
| AP0536/4/04 | 100% xanthan gum (2% w/v) aq. solution | 50 mg/g |
| AP0536/7/01 | 100% xanthan gum (1% w/v) aq. solution | 50 mg/g |
| AP0536/4/06 | 100% methylcellulose (2% w/v) aq. | 50 mg/g |
| | Solution | |
| AP0536/7/06 | 100% methylcellulose (1% w/v) aq. | 50 mg/g |
| | Solution | |
| AP0536/12/05 | 80% w/w P188 (34% w/w) aq. solution | 50 mg/g |
| | 20% w/w labrasol ALF | |
| AP0536/12/06 | 80% w/w P188 (34% w/w) aq. solution | 50 mg/g |
| | 20% w/w transcutol HP | |
| AP0536/12/07 | 80% w/w P188 (34% w/w) aq. Solution | 50 mg/g |
| | 20% w/w isopropyl myristate | |
| AP0536/16/01 | 70% w/w P188 (20% w/w) aq. solution | 50 mg/g |
| | 21.6% w/w labrasol ALF | |
| | 5.4% w/w kolliphor EL | |
| | 3% w/w isopropyl myristate | |
| AP0536/17/01 | 80% w/w P188 (30% w/w) aq. solution | 50 mg/g |
| | 20% w/w labrasol ALF | |
| AP0536/17/02 | 72% w/w P188 (20% w/w) aq. solution | 50 mg/g |
| | 10% w/w tefose 63 | |
| | 10% w/w paraffin oil | |
| | 8% w/w labrasol ALF | |
| AP0536/17/03 | 82% w/w P188 (20% w/w) aq. solution | 50 mg/g |
| | 15% w/w labrasol ALF | |
| | 2% w/w tefose 63 | |
| | 1% w/w paraffin oil | |

### Preparation of Bi-Gels Formulations

### Xanthan Gum Based Bi-Gels

A hi-gel is composed of an aqueous phase and an oily phase. Xanthan gum 1% w/v (prepared as described in table 5) was used as the aqueous phase. The oily phase was prepared by mixing labrafac lipophile WL 1349 (95% w/w) and Emulfree duo (5% w/w) at RT. In a 4 mL vial, 50 mg of PALA were accurately weighed. To this 800 mg of aqueous phase was added. The vial was left stirring at RT until complete solubilisation of the API. 150 mg of oily phase was then slowly poured in the gelled aqueous phase and mixed with the homogeniser at 5000 rpm for 5 minutes at room temperature until a homogeneous emulsion was obtained,

### Poloxamer 188-Based Bi-Gels

Poloxamer 188 (P188) 34% w/w (table 5) was used as the aqueous phase. The oily phase was prepared by mixing labrafac lipophile WL 1349 (95% w/w) and emulfree duo (5% w/w) at RT. In a 4 mL vial, 50 mg of PALA were accurately weighed. To this 800 mg of aqueous phase was added. The vial was left stirring at RT until complete solubilisation of the API. 150 mg of oily phase was then slowly poured in the gelled aqueous phase and mixed with the homogeniser at 5000 rpm for 5 minutes at room temperature until a homogeneous emulsion was obtained.

TABLE 6
List of bi-gels formulations tested in Franz Cells

| Formulation | | PALA concentration |
|---|---|---|
| code | Vehicle composition | in the formulations |
| AP0536/12/04 | Aqueous phase: 84% w/w | 50 mg/g |
| P188 | 100% P188 (34% w/w) aq. | |
| | Solution | |
| | Oily phase: 16% w/w | |
| | 95% w/w labrafac | |
| | lipophile WL 1349 | |
| | 5% w/w emulfree duo | |
| AP0536/7/02 | Aqueous phase: 84% w/w | 50 mg/g |
| Xanthan Gum | 100% xanthan gum (1% w/v) aq. | |
| | Solution | |
| | Oily phase: 16% w/w | |
| | 95% w/w labrafac | |
| | lipophile WL 1349 | |
| | 5% w/w emulfree duo | |

### In Vitro Diffusion Cells (Franz Cells) of Hydrogels

In vitro diffusion test (Franz cells, Copley) was performed to mimicking in-vivo conditions of PALA (CMI-34-1) transfer from the formulation through the skin. The test was performed in sink conditions. 100 μL of PALA (CMI-34-1)-loaded hydrogels or bi-gel (containing 5 mg of API) were added to the donor chamber of the Franz cells. 7 mL of PBS pH 7.4 was added to the receptor chamber. The chambers were separated by a Strat-M Membrane (Transdermal Diffusion Test Model, Merck) of 25 mm diameter. The Franz cells were incubated at 37° C. in an appropriate heating block and under stirring at 450 rpm. At selected time points (15, 30, 60, 120, 180, and 1440 minutes), 100 μL of samples was withdrawn from the receptor chamber and analysed by HPLC for drug release. The same volume was replaced with fresh PBS buffer pre-heated at 37° C.

### HPLC Assay of the Formulations

Assay of the formulations was performed in duplicate by UV-HPLC analysis. Using a positive displacement pipette, 300 μL of formulation was sampled and transferred to an Eppendorf and centrifuged at 13,000 rpm for 5 minutes. 100 μL supernatant was accurately weighed in a 5 mL volumetric flask filled with type I water 100%. The volumetric flasks were then vortexed for 10 seconds and sonicated in an ultrasonic bath for 5 minutes before being analysed by UV-HPLC.

### 9-Week Stability Study

A 9-week stability study was performed on the lead formulation (AP0536/23/01) stored at RT and refrigerated conditions (2-8° C.). Formulations were stored in 4 mL clear glass vial. At defined timepoints (0, 7, 14, and 63 days) samples were visually inspected (colour and appearance) and analysed by UV-HPLC. At each timepoint, 300 μL of formulation was sampled and transferred to an Eppendorf and centrifuged at 13,000 rpm for 5 minutes. 100 μL supernatant was accurately weighed in a 5 mL volumetric flask filled with type I water 100%. The volumetric flasks were then vortexed for 10 seconds and sonicated in an ultrasonic bath for 5 minutes before being analysed by UV-HPLC. The assay was performed in duplicate.

### Preparation for PK Studies

Lead formulation (AP0536/23/01) was freshly re-prepared at 3 different drug loads (0, 2, and 4% w/w PALA). Poloxamer 188 20% w/w aq. solution was prepared at a larger scale (30 g P188+120 g type I water) following the same procedure reported in Table 4. The placebo vehicle (AP0539/29/01) was prepared at 150 g scale as following:
105.0 g P188 20% w/w aq. solution
32.4 g labrasol ALF
8.1 g kolliphor EL
4.5 g isopropyl myristate
   2% w/w PALA formulation (AP0539/29/02) was prepared at 42 g scale by accurately weighing 0.84 g PALA and adding 41.160 g vehicle (AP0539/29/01). 4% w/w PALA formulation (AP0539/29/02) was prepared at 42 g scale by accurately weighing 1.68 g PALA and adding 40.32 g vehicle (AP0539/29/01).

### Results and Discussions

### Hydrogel's Investigation

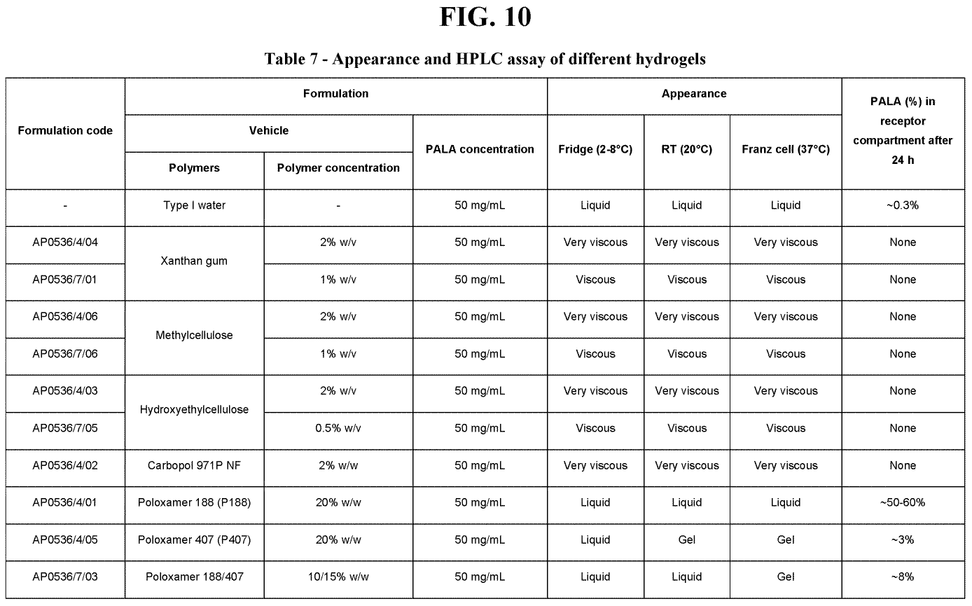

This work investigated the development of suitable preclinical formulations for compound PALA (CMI-34-1) following topical administration. Different polymers at different concentrations were investigated to prepare hydrogels, i.e. xanthan gum, methylcellulose, hydroxyethylcellulose, carbopol 971P NF, and poloxamer 188 (P188) and poloxamer 407 (P407). As a control, PALA prepared in type I water was also tested in the Franz cells. The control (water only) showed almost no PALA permeation (~0.3%) detected in the HPLC analysis after 24 hours (Table 7, FIG. 10). Xanthan gum, methylcellulose, hydroxyethylcellulose, and Carbopol 971P NF hydrogels initially prepared at 2% w/w polymer content resulting in very viscous gels. The hydrogels were loaded with PALA at 50 mg/g concentration, and the diffusion of PALA from the hydrogels through the transdermal diffusion membrane was monitored in the Franz cells.

No permeation of PALA through the membrane was observed after 24 hours (Table 7, FIG. 10). Following these results, the polymer content of the hydrogels was lowered from 2% to 1-0.5% to reduce the viscosity of the formulation and eventually facilitate the permeation of the PALA through the membrane. Unfortunately, absence of release was observed even at lower polymer concentration (Table 7, FIG. 10), except for poloxamer 188 hydrogel which showed up to 60% PALA permeated after 24 hours (Table 7, FIG. 10). As a result, P188 hydrogel was selected for further investigation.

Since poloxamer 188 at 20% showed a low viscosity, increasing concentration of P188 were investigated to provide a hydrogel with a greater viscosity that will remain firm on the skin after application. After few trials, 34% was identified to provide an acceptable viscosity, therefore, improving the adherence to the skin (Table 8). Hence, 34% w/w P188 was prepared and showed 49.7% of PALA released after 24 hours.

TABLE 8
Apperance and assay of P188 at different polymer concentration

| | | | Appearance | | | |
|---|---|---|---|---|---|---|
| | | | | | Donor | |
| | | | | | compartment | PALA (%) in |
| | Formulation | | | | of | receptor |
| Formulation | | PALA | Fridge | RT | Franz cell | compartment |
| code | Vehicle | concentration | (2-8° C.) | (20° C.) | (37° C.) | after 24 h |
| AP0536/4/01 | 100% P188 (20% | 50 mg/g | Liquid | Liquid | Liquid | ~50-60% |
| | w/w) aq. solution | | | | | |
| AP0536/7/04 | 100% P188 (40% | 50 mg/g | Viscous | Gel | Gel | None |
| | w/w) aq. solution | | liquid | | | |
| AP0536/13/01 | 100% P188 (34% | 50 mg/g | Liquid | Viscous | Viscous | 49.7% |
| | w/w) aq. solution | | | liquid | liquid | |

Further optimisation of the 34% w/w P188 formulations was carried out by incorporating different permeation enhancers, i.e. labrasol ALF, transcutol HP, and isopropyl myristate at 20% w/w, the details on composition and visual observations is provided in Table 9. The isopropyl myristate was also chosen for being a renowned emollient and texture enhancer in cosmetics. Aside from hydrogels, bi-gels were also investigated.

TABLE 9
Apperance and assay of hydrogels formulations with permeation enhancers and bi-gels

| | | | | Appearance | | |
|---|---|---|---|---|---|---|
| | | | | | | Donor |
| | Formulation | | | | | compartment |
| Formulation | | | PALA | Fridge | RT | of Franz cell |
| code | Type | Vehicle | concentration | (2-8° C.) | (20° C.) | (37° C.) |
| AP0536/12/05 | Hydrogel | 80% w/w P188 | 50 mg/g | — | Viscous | Viscous |
| | | (34% w/w) | | | liquid | liquid |
| | | 20% w/w | | | | |
| | | labrasol ALF | | | | |
| AP0536/12/06 | Hydrogel | 80% w/w P188 | 50 mg/g | — | Viscous | Viscous |
| | | (34% w/w) | | | liquid | liquid |
| | | 20% w/w | | | | |
| | | transcutol HP | | | | |
| AP0536/12/07 | Hydrogel | 80% w/w P188 | 50 mg/g | — | Viscous | Viscous |
| | | (34% w/w) | | | liquid | liquid |
| | | 20% w/w | | | | |
| | | isopropyl | | | | |
| | | myristate | | | | |
| AP0536/12/04 | Bi-gel | 84% w/w P188 | 50 mg/g | — | Viscous | Viscous |
| | | (34% w/w) | | | liquid | liquid |
| | | 16% w/w | | | | |
| | | labrafac | | | | |
| | | lipophile WL | | | | |
| | | 1394 + | | | | |
| | | emulfree DUO | | | | |
| | | (ratio 95/5) | | | | |
| AP0536/7/02 | Bi-gel | 84% w/w | 50 mg/g | — | Viscous | Viscous |
| | | xanthan gum | | | liquid | liquid |
| | | (1% w/w) | | | | |
| | | 16% w/w | | | | |
| | | labrafac | | | | |
| | | lipophile WL | | | | |
| | | 1394 + | | | | |
| | | emulfree duo | | | | |
| | | (ratio 95/5) | | | | |

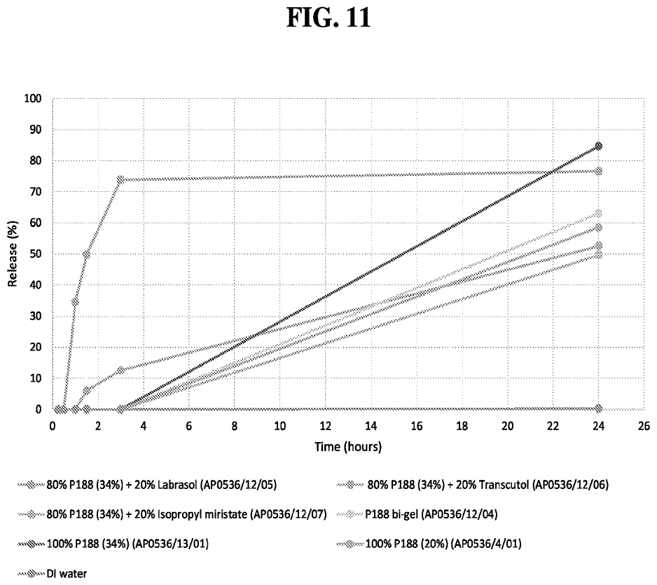

The diffusion of PALA from the formulations listed in Table 9 was tested by Franz cells. A great improvement in diffusion was observed with the addition of 20% w/w labrasol (AP0536/12/05) to the poloxamer hydrogel. The release profile (FIG. 11) showed that approx. 50% of PALA was released within 90 minutes reaching 76.6% release at 3 hours. Using P188+20% w/w transcutol (AP0536/12/06), 6.1% of PALA was released within the 90 minutes, reaching 52.5% release at 24 hours. No release of PALA was detected from the P188+20% w/w isopropyl myristate (AP0536/12/07) and P188 bi-gel (AP0536/12/04) within the 180 minutes but 49.7% and 63.1% releases were observed at 24 hours, respectively. No release within 24 hours detected with the xanthan gum bi-gel (AP0536/7/02). Following these results, P188 34% w/w aq. solution (80% w/w)+labrasol ALF (20% w/w) (AP0536/12/05) was considered the lead formulation and selected for further investigation (improved permeability and texture).

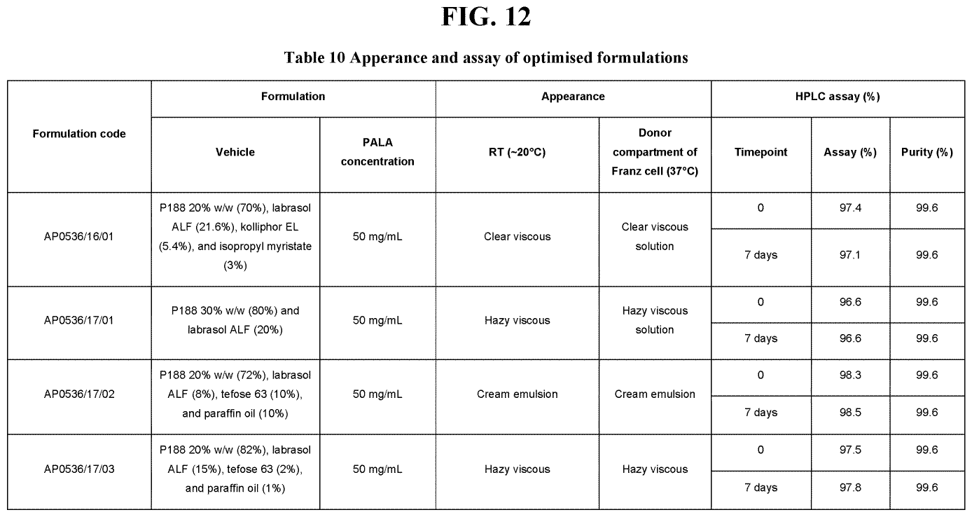

To further improve permeability and texture of the formulation (AP0536/12/05), additional components were added such as kolliphor EL, isopropyl myristate, tefose 63, and paraffin oil. To compensate the increase in viscosity observed in the initial trials in presence of the additional excipients, the poloxamer 188 concentration was reduced from 34 to 20% w/w. Additionally, to finely adjust the viscosity of AP0536/12/05, the concentration of P188 was decreased from 34 to 30% (AP0536/17/01). Table 10 (FIG. 12) lists the composition of the optimised formulations and their stability over 7 days. From the results, all the formulations were stable after 7 days stored at RT (Table 10; FIG. 12).

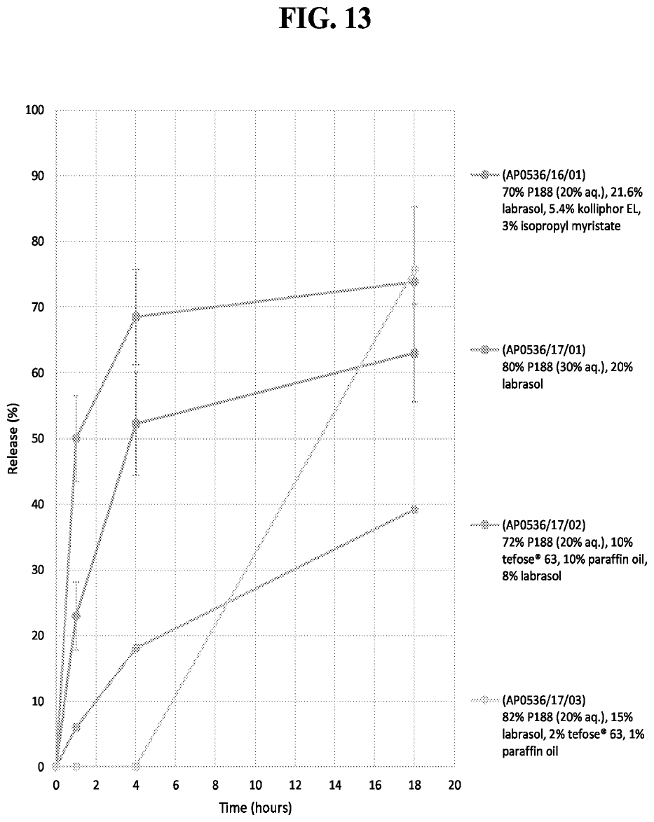

In vitro permeation studies were performed in triplicate on the selected formulation. FIG. 13 showed that after 4 hours, approx. 50% and 70% PALA was released from AP0536/16/01 and AP0536/17/01, respectively. The other two formulations, AP0536/17/02 and AP0536/17/03, released 20 and 0%, respectively, after 4 hours. Formulation comprised of 70% poloxamer 188 (20% aq. solution), 21.6% labrasol ALF, 5.4% kolliphor EL, 3% isopropyl myristate (AP0536/16/01) was finally chosen as lead to be tested in PK studies due to the great release profile and the additional presence of kolliphor EL and isopropyl myristate that might help the permeation of PALA in vivo as well providing a better texture to the formulation.

### 9-Week Stability Study

A 9 week stability study was conducted. The lead formulation including 70% P188 (20% aq.), 21.6% labrasol ALF, 5.4% kolliphor EL, 3% isopropyl myristate was freshly reprepared and stored at ambient conditions and refrigerated conditions (2-8° C.). As it can be observed in Table 11, no changes in assay and purity were detected after 9 weeks in both storage conditions.

TABLE 11
4-week stability study on lead formulation AP0536/23/01

| Formulation | | | Storage | | |
|---|---|---|---|---|---|
| number | Composition | Timepoint | condition | Assay (%) | Purity (%) |
| Standard | 1 mg/mL in Type I | — | — | — | 99.74 |
| | water | | | | |
| AP0536/23/01 | P188 20% w/w | 0 | — | 98.0 ± 1.1 | ND |
| | (70%), labrasol ALF | 1 week | Refrigerated | 99.3 ± 0.8 | ND |
| | (21.6%), kolliphor | | (2-8° C.) | | |
| | EL (5.4%), and | | RT | 99.1 ± 0.6 | ND |
| | isopropyl | 2 weeks | Refrigerated | 97.4 ± 0.6 | ND |
| | myristate (3%) | | (2-8° C.) | | |
| | | | RT | 97.6 ± 0.2 | ND |
| | | 9 weeks | Refrigerated | 99.1 ± 3.4 | 99.66 |
| | | | (2-8° C.) | | |
| | | | RT | 98.3 ± 4.2 | 99.67 |

### Preparation of Formulation for Supply to Support PK Studies

Formulation including 70% P188 (20% aq.), 21.6% labrasol ALF, 5.4% kolliphor EL, 3% isopropyl myristate was freshly reprepared with 0% w/w (AP0536/29/01), 2% w/w (AP0536/29/02) and 4% w/w (AP0536/29/03) PALA content for PK studies. HPLC assay was conducted and the results are reported in Table 12.

TABLE 12
Assay at T0 of the formulation prior shipping for PK studies

| | Formulation | | | | |
|---|---|---|---|---|---|
| | | PALA | HPLC assay (%) | | |
| Formulation | | concen- | Time- | Assay | Purity |
| code | Vehicle | tration | point | (%) | (%) |
| AP0536/29/01 | P188 20% w/w | 0 mg/mL | 0 | — | — |
| AP0536/29/02 | (70%), labrasol | 20 mg/mL | 0 | 98.1 ± | 99.67 |
| | ALF (21.6%), | | | 0.1 | |
| | kolliphor | | | | |
| AP0536/29/03 | EL (5.4%), and | 40 mg/mL | 0 | 97.8 ± | 99.67 |
| | isopropyl | | | 0.1 | |
| | myristate (3%) | | | | |

Hydrogel formulation comprising of Poloxamer 188 20% w/w (70%), labrasol ALF (21.6%), kolliphor EL (5.4%), and isopropyl myristate (3%) was selected as lead formulation to perform PK studies. This formulation was developed with the combination of poloxamer 188 hydrogel and labrasol ALF (permeation enhancer) that showed great permeability of PALA in in vitro test. Further addition of kolliphor EL and isopropyl myristate added emollient properties to the formulation and improved the texture. The formulation was physically and chemically stable for at least 9 weeks.

### In Vivo Results

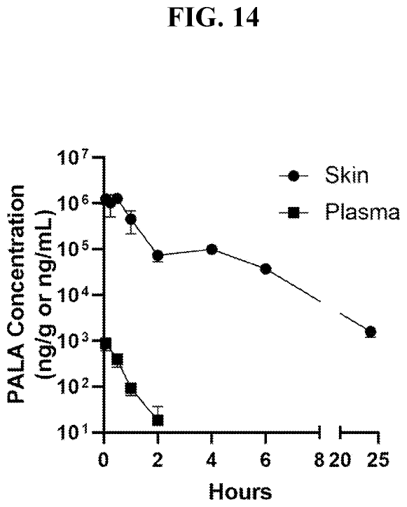

FIG. 14. Skin tissue and plasma levels of PALA over 24 h after a single topical application of formulation AP0536/29/03 4% (w/v) PALA topical to mice. Male CD-1 mice (n=3/time point) had 10 uL of 4% PALA applied to dorsal skin (final dose applied=0.4 mg/kg). Plasma and skin tissue samples were collected at 5 min, 15 min, 30 min, 1 h, 2 h, 4 h, 6 h, and 24 h post-administration and PALA levels determined by LC-MS. Mean±SEM graphed.

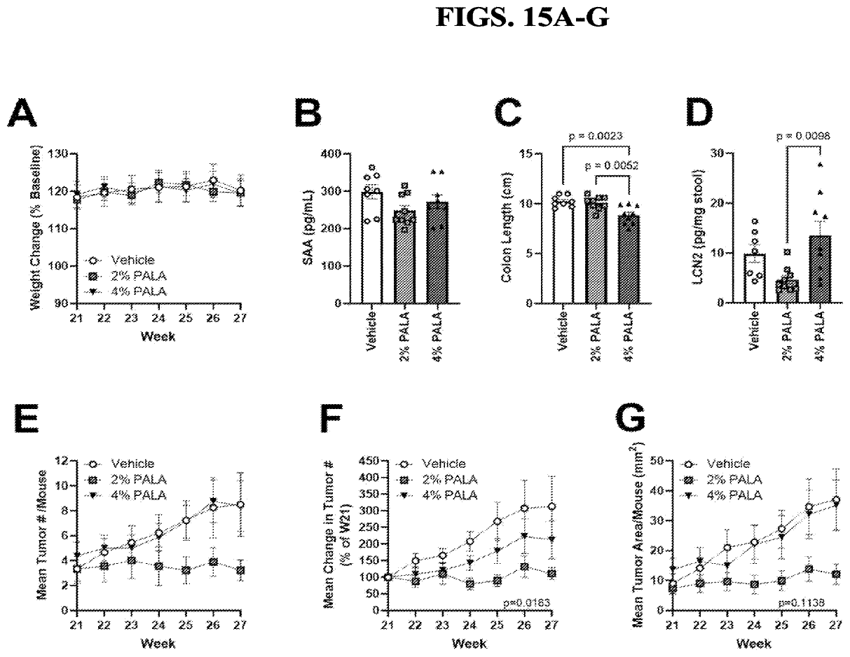

FIG. 15. Reformulated topical 2% PALA is non-toxic and effective in reducing tumor burden and total tumor area. SKH1-Elite female mice were UV irradiated thrice a week for 20 weeks to induce skin cancer. Mice were then randomized into treatment groups and treated topically with vehicle (AP0536/29/01), 2% (w/v) PALA (AP0536/29/02), or 4% (w/v) PALA (AP0536/29/03) daily for 6 weeks. A. Weight change during the treatment period presented as percentage of baseline weight. B. Levels of the liver and systemic inflammation marker serum amyloid A (SAA) at endpoint. C. Measurements of colon length at endpoint as a gross measure of intestinal pathology. D. Levels of fecal lipocalin-2 (LCN2) at endpoint as a measure of intestinal inflammation. E. Quantitation of mean tumor burden per mouse during the treatment period. F. Mean change in tumor burden per mouse during the treatment period as a percentage of the original tumor number. G. Quantification of mean tumor surface area per mouse from photographs during the treatment period. Means±SEM, n=8-9/group. Significance determined by mixed-effects model (REML) with Bonferroni's multiple comparisons test or one-way ANOVA with Tukey's multiple comparisons test. All p values <0.05 are shown on the graphs.

### REFERENCES

1. Bashline, B. Skin Cancer: Squamous and Basal Cell Carcinomas. FP essentials 481, 17-22 (2019).
2. Sang, Y. & Deng, Y. Current insights into the epigenetic mechanisms of skin cancer. Dermatologic therapy 32, e12964 (2019).
3. Linares, M. A., Zakaria, A. & Nizran, P. Skin cancer. Primary care: Clinics in office practice 42, 645-659 (2015).
4. Rogers, H. W., Weinstock, M. A., Feldman, S. R. & Coldiron, B. M. Incidence Estimate of Nonmelanoma Skin Cancer (Keratinocyte Carcinomas) in the U.S. Population, 2012. JAMA dermatology 151, 1081-1086 (2015).
5. Mansouri, B. & Housewright, C. D. The Treatment of Actinic Keratoses—The Rule Rather Than the Exception. JAMA dermatology 153, 1200-1200 (2017).
6. Lim, H. W., et al. The burden of skin disease in the United States. J Am Acad Dermatol 76, 958-972 e952 (2017).
7. The Surgeon General's Call to Action to Prevent Skin Cancer. (U.S. Dept of Health and Human Services Office of the Surgeon General, Washington, D C, 2014).
8. Fuchs, A. & Marmur, E. The kinetics of skin cancer: progression of actinic keratosis to squamous cell carcinoma. Dermatologic surgery 33, 1099-1101 (2007).
9. Del Regno, L., Catapano, S., Di Stefani, A., Cappilli, S. & Peris, K. A Review of Existing Therapies for Actinic Keratosis: Current Status and Future Directions. Am J Clin Dermatol 23, 339-352 (2022).
10. Collins, K. D. & Stark, G. R. Aspartate transcarbamylase. Interaction with the transition state analogue N-(phosphonacetyl)-L-aspartate. J Biol Chem 246, 6599-6605 (1971).
11. Grem, J. L., King, S. A., O'Dwyer, P. J. & Leyland-Jones, B. Biochemistry and clinical activity of N-(phosphonacetyl)-L-aspartate: a review. Cancer Res 48, 4441-4454 (1988).
12. Anand, S., et al. Fluorouracil enhances photodynamic therapy of squamous cell carcinoma via a p53-independent mechanism that increases protoporphyrin IX levels and tumor cell death. Molecular cancer therapeutics 16, 1092-1101 (2017).
13. Schneider, C. A., Rasband, W. S. & Eliceiri, K. W. NIH Image to ImageJ: 25 years of image analysis. Nature Methods 9, 671-675 (2012).
14. Swindell, W. R., et al. Imiquimod has strain-dependent effects in mice and does not uniquely model human psoriasis. Genome medicine 9, 24 (2017).
15. Scholzen, T. & Gerdes, J. The Ki-67 protein: From the known and the unknown. 182, 311-322 (2000).
16. Jatana, S., et al. Pyrimidine synthesis inhibition enhances cutaneous defenses against antibiotic resistant bacteria through activation of NOD2 signaling. Sci Rep 8, 8708 (2018).
17. Strober, W. & Watanabe, T. NOD2, an intracellular innate immune sensor involved in host defense and Crohn's disease. Mucosal Immunol 4, 484-495 (2011).
18. Yang, B., et al. Significance of LL-37 on Immunomodulation and Disease Outcome. BioMed research international 2020, U.S. Pat. No. 8,349,712 (2020).
19. Richmond, A. L., et al. The nucleotide synthesis enzyme CAD inhibits NOD2 antibacterial function in human intestinal epithelial cells. Gastroenterology 142, 1483-1492. e1486 (2012).
20. Collins, K. D. & Stark, G. R. Aspartate transcarbamylase: Interaction with the transition state analogue N-(phosphonacetyl)-L-aspartate. Journal of Biological Chemistry 246, 6599-6605 (1971).
21. Richmond, A. L., et al. The nucleotide synthesis enzyme CAD inhibits NOD2 antibacterial function in human intestinal epithelial cells. Gastroenterology 142, 1483-1492 e1486 (2012).
22. Erlichman, C. An overview of the clinical pharmacology of N-phosphonacetyl-L-aspartate (PALA), a new antimetabolite. Cancer Chemo- and Immunopharmacology, 65-71 (1980).
23. O'Connell, M. J., et al. Clinical trial of sequential N-phosphonacetyl-L-aspartate, thymidine, and 5-fluorouracil in advanced colorectal carcinoma. Journal of Clinical Oncology 2, 1133-1138 (1984).
24. Love, W. E., Bernhard, J. D. & Bordeaux, J. S. Topical imiquimod or fluorouracil therapy for basal and squamous cell carcinoma: a systematic review. Archives of dermatology 145, 1431-1438 (2009).
25. Griffin, M. E., Hespen, C. W., Wang, Y. C. & Hang, H. C. Translation of peptidoglycan metabolites into immunotherapeutics. Clinical & translational immunology 8, e1095 (2019).
26. Talmadge, J. E., et al. Therapy of Autochthonous Skin Cancers in Mice with Intravenously Injected Liposomes Containing Muramyltripeptidel. Cancer Research 46, 1160-1163 (1986).
27. Nardin, A., Lefebvre, L. M., Labroquere, K., Faure, O. & Abastado, P. J. Liposomal Muramyl Tripeptide Phosphatidylethanolamine: Targeting and Activating Macrophages for Adjuvant Treatment of Osteosarcoma. Current Cancer Drug Targets 6, 123-133 (2006).
28. Guryanova, S. V. & Khaitov, R. M. Strategies for Using Muramyl Peptides—Modulators of Innate Immunity of Bacterial Origin—in Medicine. Front Immunol 12, 607178 (2021).
29. Griffin, M. E., et al. Enterococcus peptidoglycan remodeling promotes checkpoint inhibitor cancer immunotherapy. Science (New York, N.Y.) 373, 1040-1046 (2021).
30. Sauder, D. N. Immunomodulatory and pharmacologic properties of imiquimod. Journal of the American Academy of Dermatology 43, S6-S11 (2000).
31. Perry, C. M. & Lamb, H. M. Topical imiquimod. Drugs 58, 375-390 (1999).
32. Asano, J., et al. Nucleotide oligomerization binding domain-like receptor signaling enhances dendritic cell-mediated cross-priming in vivo. J Immunol 184, 736-745 (2010).
33. Corridoni, D., et al. NOD2 and TLR2 Signal via TBK1 and PI31 to Direct Cross-Presentation and CD8 T Cell Responses. Front Immunol 10, 958 (2019).
34. Cooney, R., et al. NOD2 stimulation induces autophagy in dendritic cells influencing bacterial handling and antigen presentation. Nat Med 16, 90-97 (2010).
35. Whiteside, T. L. Immune responses to malignancies. Journal of Allergy and Clinical Immunology 125, S272-S283 (2010).
36. Mantovani, A. & Sica, A. Macrophages, innate immunity and cancer: balance, tolerance, and diversity. Current opinion in immunology 22, 231-237 (2010).
37. Ruffell, B. & Coussens, L. M. Macrophages and therapeutic resistance in cancer. Cancer cell 27, 462-472 (2015).
38. Pathria, P., Louis, T. L. & Varner, J. A. Targeting tumor-associated macrophages in cancer. Trends in immunology 40, 310-327 (2019).
39. Wahl, L. M. & Kleinman, H. K. Tumor-associated macrophages as targets for cancer therapy. Vol. 90 1583-1584 (Oxford University Press, 1998).
40. Andersen, M. H., Schrama, D., thor Straten, P. & Becker, J. C. Cytotoxic T cells. Journal of Investigative Dermatology 126, 32-41 (2006).
41. Najafi, M., et al. Tumor microenvironment: Interactions and therapy. Journal of cellular physiology 234, 5700-5721 (2019).
42. Reisfeld, R. A. The tumor microenvironment: a target for combination therapy of breast cancer. Critical Reviews™ in Oncogenesis 18(2013).

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating precancerous and/or cancerous cells of a subject comprising:
applying a composition topically to a bodily area of a subject that has precancerous and/or cancerous cells, and/or is adjacent to underlying precancerous or cancerous cells, or
providing said composition to said subject such that said subject is able to apply said composition to said bodily area,
wherein said composition comprises N-phosphonacetyl-L-aspartate (PALA) and water, and optionally further comprises at least one non-ionic surfactant.

2. The method of claim 1, wherein said PALA is present in said composition at a concentration of about 0.1% to about 5.0% or about 1.0-8.0%.

3. The method of claim 1, wherein said area of said subject comprises skin.

4. The method of claim 1, wherein said precancerous cells comprise actinic keratosis cells.

5. The method of claim 3, wherein said cancerous cells comprise melanoma or non-melanoma skin cancer cells.

6. The method of claim 4, wherein said non-melanoma skin cancer cells are basal cell carcinoma cells or a squamous cell carcinoma cells.

7. The method of claim 1, wherein said subject is a female, wherein said area of said subject comprises a surface of said subject's vagina, and wherein said precancerous and/or cancerous cells comprise vaginal precancerous and/or cancer cells.

8. The method of claim 1, wherein said area of said subject comprises a surface of said subject's oral cavity, and wherein said precancerous and/or cancerous cells comprise oral cavity precancerous and/or cancer cells.

9. The method of claim 1, wherein said subject is a female, wherein said area of said subject comprises said subject's cervix, and wherein said precancerous and/or cancerous cells comprise cervical precancerous or cancerous cells.

10. The method of claim 1, wherein said composition further comprises a non-ionic linear copolymer, and optionally wherein said non-ionic linear copolymer comprises poloxamer 188.

11. The method of claim 1, wherein said applying said composition, or providing said composition, is repeated daily for at least 7 days, or for at least 14 days, or for at least 21 days.

12. The method of claim 9, wherein said non-ionic linear copolymer is present in said composition at about 50%-90%, about 60%-80%, about 65%-75%, or about 70%.

13. The method of any of claims 1-12, wherein said composition further comprises a non-ionic surfactant, and optionally wherein said non-ionic surfactant is present in said composition at 10%-30% or about 22%.

14. The method of claim 13, wherein said non-ionic surfactant comprises Caprylocaproyl Polyoxyl-8 glycerides, or wherein said non-ionic surfactant excipient comprises PEG-8 mono- and diesters of caprylic (C8) and capric (C10) acids with a small fraction of mono-, di- and triglycerides, or wherein said non-ionic surfactant comprises LABRASOL ALF.

15. The method of any of claims 1-14, wherein said composition further comprises a hydrogel agent, and said composition is in the form of a hydrogel.

16. The method of any of claims 1-15, wherein said composition further comprises isopropyl myristate, and optionally wherein said isopropyl myristate is present in said composition at a concentration of about 0.5%-7% or about 1%-5%, or about 3%.

17. The method of any of claims 1-16, wherein said composition further comprises a solubilizer and/or emulsifier.

18. The method of any of claims 1-17, wherein said composition further comprises macrogolglycerol ricinoleate, and optionally wherein said macrogolglycerol ricinoleate is KOLLIPHOR EL, and optionally wherein said macrogolglycerol ricinoleate is present in said composition at a concentration of about 2%-10% or about 3%-7%, or about 5%.

19. The method of any of claims 1-18, further comprising administering an immune checkpoint inhibitor to said subject.

20. The method of any of claims 1-19, wherein said subject is a human, dog, or cat.

21. The method of any of claims 1-20, wherein said composition further comprises at least one non-PALA anti-cancer agent, and optionally wherein said at least one non-PALA anti-cancer agent comprises: tirbanibulin, fluorouracil (5-FU) and/or imiquimod (IMQ).

22. The method of any of claims 1-21, wherein said composition is in the form of a cream, emulsion, skin lotion, gel, an ointment, a spray, or is present in a skin patch.

23. The method of any of claims 1-22, wherein said composition further comprises said at least one non-ionic surfactant, and optionally wherein said non-ionic surfactant comprises poloxamer 188 and/or Caprylocaproyl Polyoxyl-8 glycerides.

24. A composition comprising:
a) N-phosphonacetyl-L-aspartate (PALA);
b) water; and
c) at least one non-ionic surfactant.

25. The composition of claim 24, wherein said PALA is present in said composition at a concentration of about 0.1% to about 5.0% or about 1.0 to 8.0%.

26. The composition of any of claims 24-25, wherein said at least one non-ionic surfactant comprises a non-ionic linear copolymer.

27. The composition of claim 26, wherein said non-ionic linear copolymer comprises poloxamer 188.

28. The composition of claim 26, wherein said non-ionic linear copolymer is present in said composition at about 50%-90%, about 60%-80%, about 65%-75%, or about 70%.

29. The composition of claim 24, wherein said at least one non-ionic surfactant is present in said composition at 10%-30% or about 22%.

30. The composition of claim 24, wherein said at least one non-ionic surfactant comprises Caprylocaproyl Polyoxyl-8 glycerides, or wherein said at least one non-ionic surfactant excipient comprises PEG-8 mono- and diesters of caprylic (C8) and capric (C10) acids with a small fraction of mono-, di- and triglycerides, or wherein said at least one non-ionic surfactant comprises LABRASOL ALF.

31. The composition of any of claims 24-30, wherein said composition further comprises a hydrogel agent, and said composition is in the form of a hydrogel.

32. The composition of any of claims 24-31, wherein said composition further comprises isopropyl myristate, and optionally wherein said isopropyl myristate is present in said composition at a concentration of about 0.5%-7% or about 1%-5%, or about 3%.

33. The composition of any of claims 24-32, wherein said composition further comprises a solubilizer and/or emulsifier.

34. The composition of any of claims 24-33, wherein said composition further comprises macrogolglycerol ricinoleate, and optionally wherein said macrogolglycerol ricinoleate is KOLLIPHOR EL, and optionally wherein said macrogolglycerol ricinoleate is present in said composition at a concentration of about 2%-10% or about 3%-7%, or about 5%.

35. The composition of any of claims 24-34, wherein said composition further comprises at least one non-PALA anti-cancer agent, and optionally wherein said at least one non-PALA anti-cancer agent comprises: tirbanibulin, fluorouracil (5-FU) and/or imiquimod (IMQ).

36. The composition of any of claims 24-35, wherein said at least one non-ionic surfactant comprises poloxamer 188 and/or Caprylocaproyl Polyoxyl-8 glycerides.

37. The composition of any of claims 24-36, wherein said composition is in the form of a cream, lotion, or gel.

38. A system or kit comprising:
a) a composition comprising N-phosphonacetyl-L-aspartate (PALA) and water, and optionally at least one non-ionic surfactant; and
b) at least one of the following:
i) a dispensing container configured to dispense said composition topically to a bodily area of a subject,
ii) a topical patch configured to deliver said composition topically to said bodily area of said subject, or
iii) an immune checkpoint inhibitor.

39. The system of kit of claim 38, wherein said composition is present in said dispensing container or said topical patch.

40. The system or kit of claim 38, wherein dispensing container is present, and wherein said dispensing container is a touchless dispensing container that allows for dispensing, and rubbing in of, said composition on said bodily area such that other areas of said subject do not come into contact with said composition.

41. The system or kit of claim 40, wherein said other areas of said subject comprises said subject's hands.

42. The system or kit of claim 40, wherein said touchless dispensing container comprises a metered-dose topical applicator.

43. The system or kit of claim 40, wherein said touchless dispensing container may be selected from the group consisting of: a CLICK Metered Topical Applicator, a TOPI-CLICK applicator, a TAPEMARK unit dose semi-solid drug delivery system, MICROBRISTLE APPLICATOR (MBA™), BACK Easy Lotion Applicator, and LIQUIBAND XL skin closure system.

44. The system or kit of claim 40, wherein said dispensing container is present and comprises a tube or sachet.

45. The system or kit of claim 38, wherein said PALA is present in said composition at a concentration of about 0.1% to about 5.0% or about 1.0-8.0%

46. The system or kit of any of claims 38-45, wherein said at least one non-ionic surfactant is present and comprises a non-ionic linear copolymer.

47. The system or kit of claim 46, wherein said non-ionic linear copolymer comprises poloxamer 188.

48. The system or kit of claim 46, wherein said non-ionic linear copolymer is present in said composition at about 50%-90%, about 60%-80%, about 65%-75%, or about 70%.

49. The system or kit of claim 38, wherein said at least one non-ionic surfactant is present in said composition at 10%-30% or about 22%.

50. The system or kit of claim 38, wherein said at least one non-ionic surfactant is present and comprises Caprylocaproyl Polyoxyl-8 glycerides, or wherein said at least one non-ionic surfactant excipient comprises PEG-8 mono- and diesters of caprylic (C8) and capric (C10) acids with a small fraction of mono-, di- and triglycerides, or wherein said at least one non-ionic surfactant comprises LABRASOL ALF.

51. The system or kit of any of claims 38-50, wherein said composition further comprises a hydrogel agent, and said composition is in the form of a hydrogel.

52. The system or kit of any of claims 38-51, wherein said composition further comprises isopropyl myristate, and optionally wherein said isopropyl myristate is present in said composition at a concentration of about 0.5%-7% or about 1%-5%, or about 3%.

53. The system or kit of any of claims 38-51, wherein said composition further comprises a solubilizer and/or emulsifier.

54. The system or kit of any of claims 38-53, wherein said composition further comprises macrogolglycerol ricinoleate, and optionally wherein said macrogolglycerol ricinoleate is KOLLIPHOR EL, and optionally wherein said macrogolglycerol ricinoleate is present in said composition at a concentration of about 2%-10% or about 3%-7%, or about 5%.

55. The system or kit of any of claims 38-53, wherein said composition further comprises at least one non-PALA anti-cancer agent, and optionally wherein said at least one non-PALA anti-cancer agent comprises: tirbanibulin, fluorouracil (5-FU) and/or imiquimod (IMQ).

56. The system or kit of any of claims 38-55, wherein said at least one non-ionic surfactant comprises poloxamer 188 and/or Caprylocaproyl Polyoxyl-8 glycerides.

57. The system or kit of any of claims 38-56, wherein said composition is in the form of a cream, lotion, or gel.
